(19) United States
(12) Patent Application Publication
(10) Pub. No.: US 2026/0103580 A1
(43) Pub. Date: Apr. 16, 2026

(54) RECYCLING RADICAL-CURED COMPOSITES WITH MATRIX RECOVERY

(71) Applicants: University of Southern California, Los Angeles, CA (US); University of Kansas, Lawrence, KS (US)

(72) Inventors: Cory Benjamin JENKINSON, Greenville, SC (US); Benjamin MILLER, Los Angeles, CA (US); Steven NUTT, Irvine, CA (US); Berl Ray OAKLEY, Lawrence, KS (US); Clarissa OLIVAR, Los Angeles, CA (US); Maria Barbara TANGALOS, Oak Park, CA (US); Clay C. WANG, Los Angeles, CA (US); Travis J. WILLIAMS, Los Angeles, CA (US); Zehan YU, Los Angeles, CA (US)

(73) Assignees: University of Southern California, Los Angeles, CA (US); University of Kansas, Lawrence, KS (US)

(21) Appl. No.: 19/357,140

(22) Filed: Oct. 14, 2025

Related U.S. Application Data
(60) Provisional application No. 63/707,006, filed on Oct. 14, 2024.

(51) Int. Cl. C08J 11/24; C08J 5/24; C12N 15/80; C12P 7/6427; D01G 11/00

(52) U.S. Cl. CPC C08J 11/24; C08J 5/243; C12N 15/80; C12P 7/6427; D01G 11/00; C08J 2325/06; C08J 2367/06; D10B 2101/12; D10B 2505/02

(57) ABSTRACT

Carbon fiber reinforced polymers (CFRPs) are increasingly replacing traditional materials in the automobile, aerospace, and energy sectors. Developing end-of-life processes that retain the value of both carbon fibers and the polymer matrix is essential. This study presents a strategy to upcycle pre- and post-consumer polystyrene-containing CFRPs, crosslinked with unsaturated polyesters or vinyl esters, into benzoic acid. The thermoset matrix is upgraded via biocatalysis using an engineered strain of Aspergillus nidulans, producing valuable secondary metabolites, such as (2Z,4Z,6E)-octa-2,4,6-trienoic acid. The process preserves much of the carbon fiber sizing, enabling the fibers to be remanufactured into new composite coupons with mechanical properties comparable to virgin substrates. This represents the first system to reclaim significant value from both the fiber and polymer matrix of CFRPs.

### CROSS-REFERENCE TO RELATED APPLICATIONS

This application claims the benefit of U.S. provisional application Ser. No. 63/707,006 filed Oct. 14, 2024, the disclosure of which is hereby incorporated in its entirety by reference herein.

### STATEMENT REGARDING FEDERALLY SPONSORED RESEARCH OR DEVELOPMENT

This invention was made with government support under Grant No(s). R21AI15623 and S10 RR025432, awarded by the National Institutes of Health, and Grant No(s). CMMI-2227649, CMMI-2134658, CHE-2018740, DBI-0821671, and CHE-0840366, awarded by the National Science Foundation. The government has certain rights in the invention.

### SEQUENCE LISTING

The text file USC0383PUSP_Sequences.xml of size 40,469 bytes created Oct. 11, 2025, filed herewith, is hereby incorporated by reference.

### TECHNICAL FIELD

In at least one aspect, the present invention is related to a method for recycling fiber-reinforced polymers, and in particular carbon-reinforced polymers (CFRPs), by using a combination of chemical and biocatalytic processes to recover both the carbon fibers and the polymer matrix is provided.

### BACKGROUND

Carbon fiber reinforced polymers (CFRPs) are composite materials comprising carbon fibers embedded in a polymer matrix, commonly a thermosetting polymer such as an epoxy,¹⁻³ unsaturated polyester,^{4,5} or vinyl ester⁶ system crosslinked with vinyl monomers, the latter acting as a reactive diluent, processing aid, and catalyst.^{7,8} CFRPs are attractive manufacturing materials in the energy, aerospace, and automotive sectors^{1-3,9} because of their high strength- and stiffness-to-weight ratios and resistance to corrosion, moisture, and chemical pollutants.¹⁰ CFRPs are generally landfilled at end-of-life because the strong three-dimensional crosslinking network of the polymer matrix makes recycling difficult and repairs impractical.^{1-3,9} By 2030, 6,000-8,000 CFRP commercial aircraft will reach their end of service,11 and by 2050, the retirement of wind turbines will have generated 483,000 tons of CFRP waste, while the demand for carbon fibers and CFRPs is projected to reach 190 kilotons.^{10,11} It is therefore imperative to establish viable recycling methods for these composites.

Current options for CFRP recycling include mechanical, thermal, and chemical processes. Mechanical recycling involves shredding CFRPs into smaller pieces, which destroys the value of the continuous fiber. Thermal recycling involves energy-intensive pyrolysis (≥450° C.), where the polymer matrix is discarded as valueless oils and gases, and the high temperatures degrade the fibers' mechanical properties. Emerging chemical recycling methods, mainly solvolysis of epoxy systems, are promising as they can recover valuable chemicals from the matrix while preserving the fiber's architecture and mechanical properties.^{1, 12}

Accordingly, there is a need for a recycling method that can recover both the carbon fibers and the polymer matrix from CFRPs without damaging their properties, while also producing useful chemical products, to reduce the growing waste from industries like aerospace, energy, and automotive.

### SUMMARY

In at least one aspect, an upcycling chemistry new to the composite space, using tandem chemical and biocatalysis to cleave and upgrade a polyolefin-containing composite matrix into high-value products is provided. It is the first approach to use a waste composite matrix as a manufacturing substrate for fine chemicals.

In another aspect, a method for recycling fiber-reinforced polymers (RPPs), and in particular, carbon fiber-reinforced polymers (CFRPs), is provided. The method includes the following steps. First, a carbon fiber-reinforced polymer, which includes carbon fibers embedded in a polymer matrix, is provided. The next step involves pre-treating the CFRP at a first temperature by soaking it in a solution containing benzyl alcohol and a phosphate base. This step causes the polymer matrix to swell, facilitating further processing. After pre-treatment, a chemical digestion process is applied to cleave the polymer matrix using a mixture of manganese (II) and cobalt(II) nitrate salts. Following the digestion, the carbon fibers and polymer degradation products are recovered from the matrix. Finally, the polymer degradation products are upgraded into secondary metabolites (e.g., valuable chemical compounds) through biocatalysis.

In another aspect, the degradation products are subjected to a fungal biocatalytic conversion process using an Aspergillus nidulans strain to produce secondary metabolites.

In another aspect, the fiber-reinforced polymers are carbon fiber-reinforced polymers (CFRPs).

In another aspect, the fiber-reinforced polymers are carbon fiber-reinforced polymers (CFRPs).

In another aspect, the valuable chemical compound produced by biocatalysis is (2Z,4Z,6E)-octa-2,4,6-trienoic acid (OTA).

In another aspect, the Aspergillus nidulans strain used in the fungal biocatalytic conversion process is genetically engineered to enhance secondary metabolite production by inserting the promoter of AN11489 (mtnA) to constitutively express AN1029 (afoA), whose gene product AfoA is a strong transcription factor and placing AN11191 (alnA) and AN11199 (alnB) genes, which encode proteins responsible for synthesis of (2Z,4Z,6E)-octa-2,4,6-trienoic acid (OTA), under control of AN1036 (afoG) promoter [afoG(p)] and the AN1034 (afoE) promoter [afoE(p)], respectively. Characteristically, AfoA binds to afoG(p) and afoE(p) to drive the transcription of alnA and alnB. A positive feedback loop is created by inserting a copy of afoA under the control of the afoE promoter, thereby enhancing afoA transcription and promoting OTA biosynthesis.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1. Top: proposed reaction for CFRP digestion. Bottom: resulting carbon fibers from the digestion of pre-consumer CFRPs. Reaction conditions were optimized to obtain clean carbon fiber plies that retained their plain weave pattern, which are summarized in Table 4.

FIGS. 2A and 2B. (A) Schematic representation of the LO11055 genetic design and biosynthesis of OTA. The promoter of AN11489, which we identify as a metallothionein gene and hereby designate mtnA, was inserted to constitutively express AN1029 (afoA), whose gene product, AfoA, is a strong transcription factor. AN11191 (alnA) and AN11199 (alnB), which produce AlnA and AlnB, the two proteins responsible for OTA synthesis, were then placed under the control of the AN1036 (afoG) promoter [afoG(p)] and the AN1034 (afoE) promoter [afoE(p)], respectively. AfoA binds to these promoters to drive transcription of alnA and alnB. Additionally, a copy of afoA under control of afoE(p) was inserted, creating a positive feedback loop, in which afoE(p) activates additional afoA transcription. (B) Comparative OTA production by type of medium where MM: minimal medium, LMM: lactose minimal medium, GMM: glucose minimal medium, and BAMM: benzoic acid minimal medium. OTA yields were quantified using HPLC-DAD. Bars represent means and error bars represent standard deviations. *p<0.05.

FIG. 3. Apparatus setup to enable continuous oxygen delivery to the Parr reactor.

FIG. 4. Purification of benzoic acid from composite digest where CFRP: carbon fiber reinforced polymer; CF: carbon fiber; EA: ethyl acetate; HCl: hydrochloric acid diluted in DI water (pH 2); and CHCl3: chloroform.

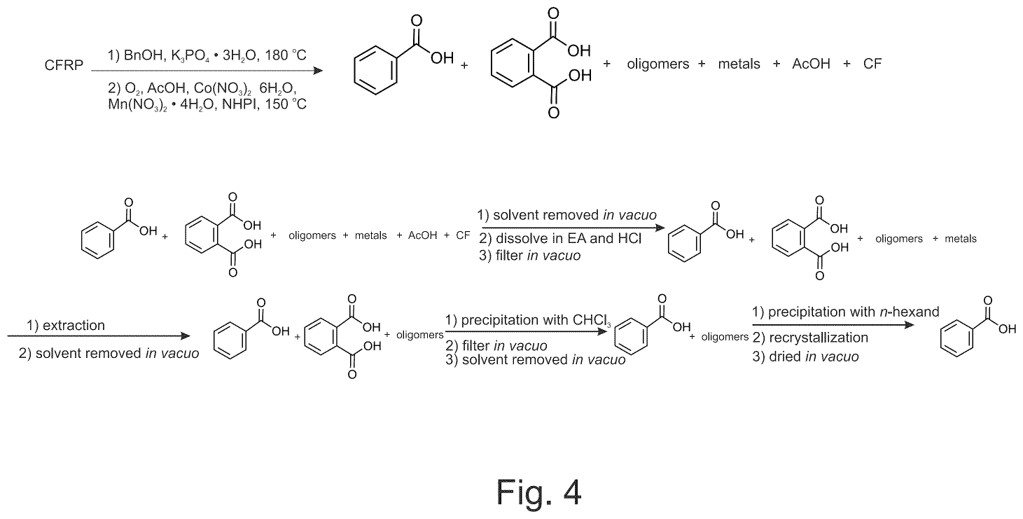

FIGS. 5A and 5B. SEM images of virgin carbon fibers at (a) 1,000× and (b) 20,000×.

FIGS. 6A and 6B. SEM images of rCF washed with H2O at (a) 1,000× (a) and (b) 20,000×

FIGS. 7A and 7B. SEM images of rCF washed with HCl pH 5 at (a) 1,000× and (b) 20,000×.

FIGS. 8A and 8B. SEM images of rCF washed with HNO3 pH 5 at (a) 1,000× and (b) 20,000×.

FIGS. 9A and 9B. SEM images of ICF washed with HNO3 pH 2 at (a) 1,000× and (b) 20,000×.

FIGS. 10A and 10B. (A) XPS survey and (B) C1S high resolution spectra of virgin carbon fibers.

FIGS. 11A and 11B. (A) XPS survey and (B) C1S high resolution spectra of recovered carbon fibers.

FIGS. 12A and 12B. (A) XPS survey and (B) C1S high resolution spectra of recovered carbon fibers treated with nitric acid.

FIG. 13. Single fiber tensile test results of virgin carbon fibers, vCF, and recovered carbon fibers, rCF.

FIG. 14. Cross section, at 200× of (top) reference 5320-1 CFRP and (bottom) second generation CFRP.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including” or “comprising.” Typically, this phrase is used to denote that an object is formed from a material.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

Throughout this application, where publications are referenced, the disclosures of these publications in their entireties are hereby incorporated by reference into this application to more fully describe the state of the art to which this invention pertains.

“Room temperature” means a temperature from 20 to 30° C.

### Abbreviations

“AcOH” means acetic acid.

“BAMM” means benzoic acid minimal medium (MM+10 g/L benzoic acid).

“BA” means benzoic acid.

“BnOH” means benzyl alcohol, a solvent used to swell and soften the polymer matrix.

“CF” means carbon fiber.

“CFs” means carbon fibers.

“CFRP” means carbon fiber-reinforced polymers.

“DI” means deionized.

“DI water” means deionized water.

“DMSO” means dimethyl sulfoxide.

“EA” means ethyl acetate.

“EGDMA” means ethylene glycol dimethacrylate.

“FRP” means fiber-reinforced polymers.

“GMM” means glucose minimal medium (MM+10 g/L glucose).

“HCl” means hydrochloric acid.

“HNO₃” means nitric acid.

“H₂O₂” means hydrogen peroxide.

“HPLC” means high-performance liquid chromatography.

“HPLC-DAD” means high-performance liquid chromatography with diode-array detection.

“K₃PO₄·3H₂O” means tripotassium phosphate trihydrate.

“KH₂PO₄” means potassium dihydrogen phosphate.

“LMM” means lactose minimal medium (MM+15 g/L lactose).

“LO11055” means the engineered Aspergillus nidulans strain used for OTA biosynthesis.

“MF” means the final mass of the recovered carbon fibers after the matrix has been degraded and the fibers have been separated (measured in grams).

“MI” means the initial mass of the composite before processing (measured in grams).

“MgSO₄·7H₂O” means magnesium sulfate heptahydrate.

“mM” means millimolar.

“MM” means minimal medium.

“MMA” means methyl methacrylate.

“NaNO₃” means sodium nitrate.

“NHPI” means N-hydroxyphthalimide, a catalyst used in oxidative reactions to help degrade the polymer matrix.

“NMR” means nuclear magnetic resonance.

“OTA” means octa-2,4,6-trienoic acid.

“PA” means phthalic acid.

“pH” means negative logarithm of hydrogen ion concentration, a measure of acidity or alkalinity.

“PKS” means polyketide synthase.

“PS” means polystyrene.

“rCF” means recovered carbon fibers, which are the fibers retrieved after the composite's polymer matrix is degraded.

“SEM” means scanning electron microscopy.

“TS” means tensile strength.

“VBO” means vacuum bag only, a curing process for composite lamination.

“vCF” means virgin carbon fiber.

“Wm” means matrix content weight percentage, calculated as the percentage of polymer matrix removed during the process.

“Wr” means resin content.

“XPS” means X-ray photoelectron spectroscopy.

To determine the “percent identity” (i.e., percent sequence identity) of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a refinement, the sequences are aligned for maximum correspondence over a specified comparison window, as measured by sequence comparison algorithms or by visual inspection. In a refinement, the length of a first sequence aligned for comparison purposes is at least 80% of the length of a second sequence and, in some embodiments, is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For purposes of the present disclosure, the comparison of sequences and determination of percent identity between two sequences can be accomplished using a Blossum 62 scoring matrix with a gap penalty of 12, a gap extend penalty of 4, and a frameshift gap penalty of 5. In this regard, the following oligonucleotide alignment algorithms may be used: BLAST (GenBank URL: www.ncbi.nlm.nih.gov/cgi-bin/BLAST/, using default parameters: Program: BLASTN; Database: nr; Expect 10; filter: default; Alignment: pairwise; Query genetic Codes: Standard (1)), BLAST2 (EMBL URL: http://www.embl-heidelberg.de/Services/index.html using default parameters: Matrix BLOSUM62; Filter: default, echofilter: on, Expect: 10, cutoff: default; Strand: both; Descriptions: 50, Alignments: 50), or FASTA, search, using default parameters. When sequences differ in conservative substitutions, the percent identity may be adjusted upwards to correct for the conservative nature of the substitution. Sequences that differ by such conservative substitutions are said to have “sequence similarity” or “similarity.” The means for making this adjustment are well known to those of skill in the art. Typically this involves scoring a conservative substitution as a partial rather than a full mismatch, thereby increasing the percentage sequence identity. In some refinements, nucleotide sequences or amino acid sequences having at least (in increasing order of preference) 70, 80, 90, 95, 97, or 99 percent identity to any nucleotide sequences or amino acid sequences referenced or provided herein can be used.

This application includes a Sequence Listing in XML format, which is incorporated herein by reference in its entirety. The Sequence Listing provides nucleic acid and amino acid sequences used to construct the engineered Aspergillus nidulans strains described herein. Specifically, SEQ ID NO: 1 discloses the AN11489 (mtnA) promoter (1,646 bases) and includes the ATG start codon for afoA; SEQ ID NO: 2 provides the AfoA protein amino acid sequence; SEQ ID NOs: 3 and 4 provide the AN1029 (afoA) coding sequence with introns (SEQ ID NO: 3) and with introns removed (SEQ ID NO: 4); SEQ ID NO: 5 provides the AN1034 promoter (useful, e.g., for driving afoE expression); SEQ ID NO: 6 provides the AN1036 promoter (useful, e.g., for driving afoG expression); SEQ ID NOs: 7 and 8 provide the AN11191 (alnA) coding sequence with introns (SEQ ID NO: 7) and with introns removed (SEQ ID NO: 8); SEQ ID NO: 9 provides the AlnA protein sequence; SEQ ID NO: 10 provides the AN11199 (alnB) protein sequence; and SEQ ID NOs: 11 and 12 provide the AN11199 (alnB) coding sequence with a single intron (SEQ ID NO: 11) and with the intron removed (SEQ ID NO: 12). In exemplary embodiments, one or more of SEQ ID NOs: 1-12 are operably linked to constitutive or inducible fungal promoters and integrated into, or maintained on, suitable vectors in A. nidulans to drive overexpression of mtnA, afoA, alnA, and alnB (and, where applicable, to regulate afoE and afoG via SEQ ID NOs: 5-6), thereby enhancing octa-2,4,6-trienoic acid (OTA) biosynthesis from benzoic-acid-derived intermediates.

The term “operably linked” refers to a functional relationship between two or more nucleic acid sequences such that one sequence (e.g., a promoter, enhancer, or regulatory element) is positioned relative to another sequence (e.g., a coding sequence or structural gene) in a manner that allows the regulatory sequence to control the transcription or, where appropriate, translation of the downstream sequence. In the context of a promoter operably linked to a coding sequence, the phrase means that the promoter is placed upstream of and in correct orientation with the coding sequence so that it can direct transcription initiation and produce the desired RNA transcript. Similarly, where a signal peptide or leader sequence is operably linked to a gene, the linkage permits proper expression and processing of the encoded polypeptide.

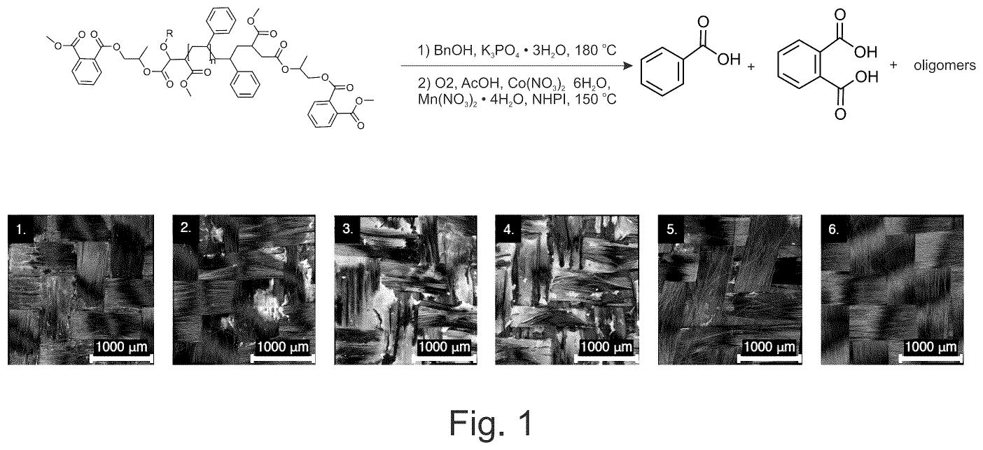

In at least one aspect, a method for recycling fiber-reinforced polymers (FRP), and in particular, carbon fiber-reinforced polymers (CFRPs) is provided. FIG. 1 depicts a proposed reaction for CFRP digestion. The method includes the following steps. First, a fiber-reinforced polymer, which includes fibers embedded in a polymer matrix, is provided. Examples of fibers include, but are not limited to, carbon fibers, glass fibers (E-glass, S-glass), aramid fibers (Kevlar, Twaron), basalt fibers, natural fibers (jute, hemp, flax, sisal), boron fibers, and alumina fibers. Carbon fibers are found to be particularly relevant. The next step involves pre-treating the FRP by soaking it in a benzyl alcohol solution and a phosphate base. Suitable phosphate bases include tripotassium phosphate trihydrate (K₃PO₄·3H₂O) and potassium dihydrogen phosphate (KH₂PO₄), which may be used individually or in combination to form a phosphate buffer system. In a refinement, the pre-treatment is at room temperature. This step causes the polymer matrix to swell, facilitating further processing. After pre-treatment, a chemical digestion process is applied to cleave the polymer matrix using a mixture of transition metal salts and in particular, manganese(II) and cobalt(II) nitrate salts. In a refinement, the digestion is performed at a temperature from 80 to 140° C. for a reaction time of 5 to 24 hours or more.

Typically, this step produces benzoic acid. Following the digestion, the fibers (e.g., carbon fibers) and polymer degradation products are recovered from the matrix. Finally, the polymer degradation products are upgraded into secondary metabolites through biocatalysis. In this step, the degradation products are subjected to a fungal biocatalytic conversion process using an Aspergillus nidulans strain to produce secondary metabolites. In a variation, the biocatalysis is performed at temperatures of 20 to 40° C. for a reaction time of 5 to 24 hours or more. In some refinements, the biocatalysis is performed at a temperature of at least 15° C., 20° C., 25° C., or 30° C., and at most 50° C., 45° C., 40° C., or 35° C. In further refinements, the biocatalysis is performed for a reaction time of at least 1 hour, 2, hours, 3 hours, 5 hours, or 10 hours and at most 48 hours, 24 hours, 20 hours, 15 hours, 10 hours, or 7 hours.

In another aspect, the reaction mixture includes transition-metal nitrate salts to promote oxidative depolymerization of resin components and facilitate recovery of carbon fibers. The metal catalyst system comprises manganese(II) nitrate tetrahydrate (Mn(NO₃)₂·4H₂O) and cobalt(II) nitrate hexahydrate (Co(NO₃)₂·6H₂O), typically independently present in concentrations of about 1 to 25 mM, such as 1 to 25 mM or 8 to 15 mM each, based on the total solvent volume. In certain variations, the molar ratio of manganese(II) to cobalt(II) nitrate is from about 1:2 to 2:1, preferably approximately 1:1, to achieve balanced redox activity. In a refinement, The reaction medium may further contain a co-catalyst such as N-hydroxyphthalimide (NHPI) in a molar ratio of about 0.5:1 to 2:1 relative to the combined transition-metal salts, and acetic acid as the solvent in a concentration sufficient to dissolve the reagents and maintain homogeneous catalytic conditions. This combination provides an efficient oxidative environment for polymer matrix degradation and recovery of carbon fibers with high tensile strength retention.

In another aspect, the secondary metabolites are valuable chemical compounds. In this context, a valuable chemical compound refers to a chemical compound that has significant commercial or industrial utility and is often sought after due to its applications in areas such as pharmaceuticals, synthetic chemistry, or advanced manufacturing. These products are considered “high-value” because they can be sold at a premium due to their usefulness in creating other valuable materials or compounds, or because they serve as critical intermediates in the production of specialty chemical. In a refinement, the valuable chemical compound produced by biocatalysis is (2Z,4Z,6E)-octa-2,4,6-trienoic acid (OTA).

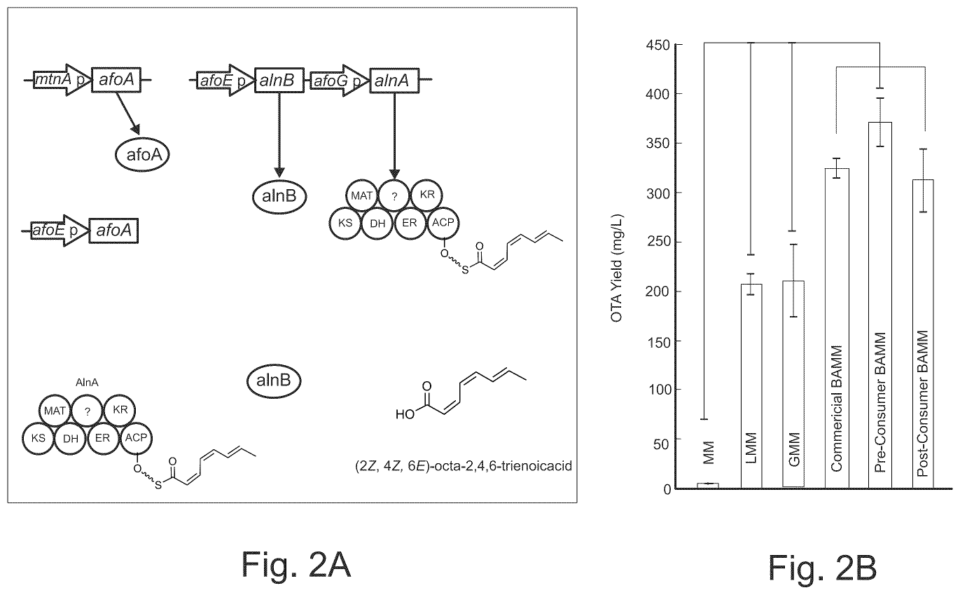

Referring to FIG. 2, the Aspergillus nidulans strain used in the fungal biocatalytic conversion process is genetically engineered to enhance secondary metabolite production by inserting the promoter of AN11489 (mtnA) to constitutively express AN1029 (afoA), whose gene product AfoA is a strong transcription factor and placing AN11191 (alnA) and AN11199 (alnB) genes, which encode proteins responsible for synthesis of (2Z,4Z,6E)-octa-2,4,6-trienoic acid (OTA), under control of AN1036 (afoG) promoter [afoG(p)] and the AN1034 (afoE) promoter [afoE(p)], respectively. Characteristically, AfoA binds to afoG(p) and afoE(p) to drive the transcription of alnA and alnB. A positive feedback loop is created by inserting a copy of afoA under the control of the afoE promoter, thereby enhancing afoA transcription and promoting OTA biosynthesis.

In another aspect, the polymer matrix is composed of a thermoplastic polymer or a thermoset polymer. Polystyrene is an example of a thermoplastic polymer. The polymer matrix is crosslinked with an unsaturated polyester or vinyl ester. Polymers crosslinked with unsaturated polyesters or vinyl esters can be formed from various vinyl monomers, which are the most common crosslinking agents. These monomers help form the three-dimensional network structure of the matrix. Styrene can be used vinyl monomer for this purpose, acting as a reactive diluent to facilitate the polymerization process. Other examples include methyl methacrylate (MMA), which enhances strength and weather resistance in the composite, and divinylbenzene (DVB), often used to add further crosslinking to improve mechanical properties and durability. Additionally, ethylene glycol dimethacrylate (EGDMA) is another crosslinker used with vinyl esters and unsaturated polyesters to form a durable polymer network. These monomers react with the unsaturated polyester or vinyl ester matrix during the curing process, creating a highly crosslinked structure that provides the composite with mechanical strength and chemical resistance. The polymer matrix binds the fibers (e.g., carbon fibers) and transfers loads between them, while the crosslinking ensures the material's rigidity and durability.

In another aspect, an Aspergillus nidulans strain is formed by inserting the promoter of AN11489 (mtnA) to constitutively express AN1029 (afoA), whose gene product AfoA is a strong transcription factor. AN11191 (alnA) and AN11199 (alnB) genes, which encode proteins responsible for synthesis of (2Z,4Z,6E)-octa-2,4,6-trienoic acid (OTA), are placed under control of AN1036 (afoG) and AN1034 (afoE) promoters, respectively, wherein AfoA binds to the afoG promoter (afoG(p)) and the afoE promoter (afoE(p)) to drive the transcription of alnA and alnB. A positive feedback loop is created by inserting a copy of afoA under the control of the afoE promoter, thereby enhancing afoA transcription and promoting OTA biosynthesis.

In another aspect, an Aspergillus nidulans strain is engineered to enhance octa-2,4,6-trienoic acid (OTA) biosynthesis by coordinated promoter substitution and feedback regulation. In this refinement, the promoter of AN11489 (mtnA) is operably linked to AN1029 (afoA) to drive its constitutive expression, wherein the gene product AfoA functions as a strong transcriptional activator. The AN11191 (alnA) and AN11199 (alnB) genes, which encode enzymes responsible for the biosynthesis of (2Z,4Z,6E)-octa-2,4,6-trienoic acid, are placed under the control of the AN1036 (afoG) and AN1034 (afoE) promoters, respectively. AfoA binds to both the afoG promoter (afoG(p)) and the afoE promoter (afoE(p)), thereby driving transcription of alnA and alnB. To further amplify pathway activation, a copy of afoA is inserted under the control of the afoE promoter, establishing a positive feedback loop that enhances afoA transcription and promotes sustained OTA production.

In another aspect, the carbon fibers obtained from the method retain at least 97% of their initial tensile strength after the recycling process. In some refinements, the carbon fibers retain at least 90%, 92%, 94%, 95%, 96%, 97%, 98%, 99%, or 99.5% of their tensile strength. In further refinements, the retained tensile strength is measured relative to the tensile strength of virgin carbon fibers prior to the recycling process. In some variations, the tensile strength retention is achieved following one, two, or multiple recycling cycles, indicating that the recycled fibers maintain substantially the same mechanical performance as the original fiber.

In another aspect, the method further includes a step of cleaning the recovered fibers (e.g., recovered carbon fibers) to form cleaned recovered fibers. The recovered fibers can be cleaned through a series of chemical treatments after the polymer matrix is degraded. Initially, the fibers are washed with solvents such as acetone or ethyl acetate to remove any remaining polymer residues or byproducts from their surface. Following this, the fibers undergo an acid treatment, often using diluted acids like hydrochloric acid (HCl) or nitric acid (HNO₃), to further clean the surface. This acid treatment helps to remove any residual sizing agents or chemical contaminants left after the degradation process. In particular, soaking the fibers in diluted nitric acid at specific pH levels (e.g., pH 2 or pH 5) restores surface functionality, such as hydroxyl and carboxyl groups. After the acid treatment, the fibers are thoroughly rinsed with deionized water to neutralize any remaining acid and remove soluble contaminants, followed by drying either in air or under vacuum to eliminate residual moisture. In some cases, sonication in water is also used to further clean the fibers by dislodging any particles or contaminants still attached to the surface. This cleaning process ensures that the recovered fibers are free of surface contamination and can be reused in manufacturing, retaining most of their mechanical properties, such as tensile strength. Additionally, sizing restoration, such as with nitric acid treatment, can improve fiber adhesion for future composite applications.

In another aspect, the recovered fibers (typically after cleaning) can be used in the manufacture of second-generation composites. In a refinement, the second-generation composites are manufactured by embedding the recovered fibers into a polymer resin to form new fiber-reinforced polymers (e.g., new carbon fiber-reinforced polymers). For example, the second-generation composites are manufactured by embedding the recovered carbon fibers into an aerospace-grade resin and curing for example by a vacuum-bag-only process.

The following examples illustrate the various embodiments of the present invention. Those skilled in the art will recognize many variations that are within the spirit of the present invention and scope of the claims.

The method is demonstrated on polyester and vinyl ester thermosets. These materials feature durability, low cost, effective fiber-matrix adhesion, and negligible composite shrinkage.¹⁰ Such materials are commonly crosslinked by vinyl monomer polymerization; the case features styrene, as it is both economical and has attractive properties, such as a low molecular weight, high reactivity, and easy processing.⁸ Polystyrene (PS)-containing composites are not limited to CFRPs;¹³ there is an increasing effort to incorporate post-consumer PS, other organics, ^{14,15} and natural fibers into composites. ^{16,17} The composite matrix cleavage proceeds in two steps: (1) presoaking the composite in benzyl alcohol and tripotassium phosphate trihydrate and (2) digestion with manganese(II) and cobalt(II) nitrate salts. CFRP panels featuring a FiberGlast Part #77 polyester molding resin were first used. To measure the composition of both materials, the matrix was first digested with sulfuric acid and hydrogen peroxide at 100° C., separating the polymer matrix from the fibers. This provides an accurate quantification of fiber versus polymer content (Tables 1-3).

TABLE 1
Summary of matrix content weight measurement of
FiberGlast Part #77 Polyester Molding Resin CFRP.

| Entry | MI (g) | MF (g) | Wm (%) |
|---|---|---|---|
| 1 | 0.6754 | 0.2464 | 63.52 |
| 2 | 0.7222 | 0.2545 | 64.76 |
| 3 | 0.8147 | 0.2466 | 69.73 |
| 4 | 0.8361 | 0.2511 | 69.97 |
| 5 | 0.8383 | 0.2456 | 70.7 |
| 6 | 0.9057 | 0.2495 | 72.45 |

TABLE 2
Summary of matrix content weight measurement of FiberGlast
Part #78 - Laminating Resin AropolTM L 67355 T-20 TS CFRP

| Entry | MI (g) | MF (g) | Wm (%) |
|---|---|---|---|
| 1 | 0.8219 | 0.2592 | 68.46 |
| 2 | 0.7016 | 0.2563 | 63.47 |
| 3 | 0.794 | 0.2536 | 68.06 |
| 4 | 0.6406 | 0.2564 | 59.98 |
| 5 | 0.6335 | 0.2579 | 59.29 |

TABLE 3
Summary of matrix content weight measurement
of post-consumer CFRP.

| Entry | MI (g) | MF (g) | Wm (%) |
|---|---|---|---|
| 1 | 1.0012 | 0.2655 | 73.48 |
| 2 | 1.0493 | 0.3114 | 70.32 |
| 3 | 1.0374 | 0.305 | 70.6 |

In the first step, composite panels (50.8×38.1 mm) are incubated in benzyl alcohol and K₃PO₄·3H₂O for 24 hours at 180° C. This appears to swell the composite and enables more facile intercalation of the reagents of the subsequent step. While a similar approach was used with neutral benzyl alcohol when cleaving epoxy composites by catalytic oxidation,^{2,12} a base is added here to cleave the polyester crosslinks. The swelling and disconnection are visible, as the pretreatment causes matrix cracking near the surface, yielding a white crystalline substance on the surface of the carbon fiber plies. Infrared spectra of the crystalline material and clear-yellow benzyl alcohol solution resulting from the pretreatment reveal that only the resulting solid contains esters.

Initial attempts at oxidative matrix cleavage (vide infra) without pretreatment involving smaller panels (25.4 mm×25.4 mm) and a catalyst loading of 4-7 wt % (relative to the composite) for metal catalysts and NHPI yield clean carbon fibers; however, with larger composites suitable for remanufacture, only partial digestion of the matrix is achieved (Table 4, entry 1), thus justifying the added step.

TABLE 4
Optimization of reaction conditions for pre-consumer
CFRPs (FiberGlast Part #77 Polyester Molding Resin
CFRPs) of dimensions 50.8 × 38.1 mm. The following are
abbreviated for clarity: K3PO4: K3PO4•3H2O; Co: cobalt
nitrate hexahydrate; and Mn: manganese nitrate tetrahydrate.
Weight percent is relative to the composite.

| | | | | | | | | Time |
|---|---|---|---|---|---|---|---|---|
| | | | | | | | | (hrs) |
| | BnOH | K₃PO₄ | Time | Co | Mn | NHPI | AcOH | dupli- |
| Entry | (mL) | (%) | (hrs) | (%) | (%) | (%) | (mL) | cate |
| 1 | — | — | — | 4 | 4 | 7 | 125 | 24 |
| 2 | 100 | — | 24 | 9 | 10 | 9 | 100 | 24 |
| 3 | 100 | 11 | 8 | 4 | 4 | 7 | 100 | 24 |
| 4 | 100 | 11 | 24 | 4 | 4 | 7 | 100 | 24 |
| 5 | 100 | 11 | 24 | 4 | 5 | 7 | 250 | 48 |
| 6 | 100 | 11 | 24 | 7 | 8 | 7 | 100 | 24 |

The solvolysis product, pretreated polymer still affixed to the fibers, is further processed with conditions introduced for converting PS to benzoic acid.¹⁸ These conditions are adapted to yield clean carbon fiber plies that retain their plain weave pattern, which are easily imaged via light microscopy (FIG. 1). Optimization of reaction conditions are summarized in Table 4. Notably, K₃PO₄·3H₂O is required for complete digestion, an observation evident when comparing entries 2 and 6 in Table 4. Increasing the reaction time and amount of acetic acid in the reaction mixture leads to more complete digestion with lower catalyst loading. However, this formulation can leave residual polymer matrix on the fibers (Table 4, entry 5). Characterization via 1H NMR reveals that all entries in Table 4 produce benzoic acid, phthalic acid, and oligomers of different chain lengths.

The reaction conditions in Table 4, entry 6 yield a clean fiber fabric and are used to isolate benzoic acid for fungal upgrading. Using these conditions, benzoic acid is isolated in yields of 93-102% relative to the polystyrene content in the composite. Although most of the benzoic acid yield is attributed to polystyrene conversion, some of the yield can be credited to the conversion of residual benzyl alcohol²² remaining in the composite after pretreatment, even with thorough washing with acetone. Similar reaction conditions are applied to post-consumer CFRPs (50.8×38.1 mm) consisting of polystyrene crosslinked with vinyl esters and chopped, unaligned carbon fibers. This results in a yield of 16-25% for benzoic acid. The complete isolated and recovered composition for pre- and post-consumer composites is summarized in Table 5.

TABLE 5
Mass characterization of reaction mixture where rCF: recovered
carbon fibers; PA: phthalic acid; and BA: benzoic acid. Pre-
consumer composite was comprised of FiberGlast Part #78 -
Laminating Resin AropolTM L 67355 T-20 TS or FiberGlast Part #77
Polyester Molding Resin. Range is provided for benzoic acid yield
in both pre- and post- consumer composites to account for
standard deviation in matrix content. The benzoic acid yield
range for the post-consumer composite additionally accounts
for 20-30% styrene content provided by the manufacturer.

| | | | | | BA |
|---|---|---|---|---|---|
| | | | Oligomers | | yield |
| Composite | rCF (g) | PA (g) | (g) | BA (g) | (%) |
| Pre-consumer | — | 0.101 | 1.051 | 1.177 | 93-102 |
| (Resin 77) | | | | | |
| Pre-consumer | 1.489 | 0.063 | 0.761 | 0.401 | 37-42 |
| (Resin 78) | | | | | |
| Post-consumer | 1.386 | 0.164 | 0.333 | 0.217 | 16-25 |

Prior to upgrading, the crude oxidation product is purified via a series of filtrations, extractions, and crystallizations. Although the final purification step of recrystallizing benzoic acid removes most of the remaining oligomers in the sample, a yellow impurity co-crystallizes with the product and is detected by elemental analysis (Table 6). The impurity does not inhibit fungal upgrading.

TABLE 6
Elemental analysis of recrystallized benzoic acid from pre-
consumer CFRP (Fiber Glast Part #77 Polyester Molding Resin).

| Element | Theoretical | Pre-consumer |
|---|---|---|
| Carbon | 68.85 | 68.02 |
| Hydrogen | 4.95 | 4.73 |

PS-derived benzoic acid was previously upgraded to secondary metabolites via the model fungal organism Aspergillus nidulans,¹⁸ wherein polymer digest served as the sole carbon source. This, in conjunction with its diverse secondary metabolite profile, makes A. nidulans the ideal candidate for the biochemical upcycling of CFRP-derived benzoic acid into a valuable fungal metabolite. In this case, the composite matrix is upgraded to (2Z,4Z,6E)-octa-2,4,6-trienoic acid (OTA). OTA is an intermediate in the biosynthetic pathway for (+)-asperlin, a molecule with antitumor, antibiotic, and anti-inflammatory properties.^{19,20} OTA is of particular interest because its conjugated backbone readily lends itself as a potential feedstock compound for synthetic chemistry applications,²¹ such as ketonization, Kolbe coupling, and olefin coupling.

Thus, the formation of a versatile platform chemical from a waste stream of industrial scale is demonstrated. In A. nidulans, the production of OTA requires the expression of two genes: alnA (AN11191 using the FungiDB.org gene designation), which encodes a polyketide synthase (PKS), and alnB (AN11199), which is thought to be required for the release of OTA from the PKS. An OTA-producing strain, LO4912, was previously developed by replacing the native promoters of alnA and alnB with a regulatable alcA promoter.²⁰ To achieve higher yields and eliminate the need for induction, a strain was engineered with a strong constitutive promoter system that employs a positive feedback loop, building on recent success with a similar system in the production of the fungal secondary metabolite asperbenzaldehyde.¹⁸ In the resultant strain, LO11055, the feedback loop promotes the expression of alnA and alnB, driving OTA biosynthesis (FIG. 2A).

LO11055 is used for the biosynthetic conversion of CFRP-derived benzoic acid into OTA. In a representative experiment, LO11055 is inoculated into liquid minimal media (MM) containing 10 g/L of glucose (GMM), 15 g/L of lactose (LMM), or 10 g/L of benzoic acid obtained commercially, isolated from a pre-consumer composite source, or isolated from a post-consumer composite source. A minimal medium without a carbon source is used as a negative control. It is found that LO11055 successfully utilizes benzoic acid from all three sources to grow and produce OTA. The OTA yield is determined via HPLC-DAD with statistical significance quantified by the unpaired t-test (FIG. 2B). Notably, three benzoic acid sources (commercial, FiberGlast Part #77-derived, and postconsumer-derived) yield OTA at statistically significant higher yields than either GMM or LMM. All carbon-source containing conditions also outperform previously reported bacterial-biosynthesized OTA yields by 172-185%.²¹ The elevated yields of OTA associated with composite-derived benzoic acid as the sole carbon source are promising results that hold up the stepwise chemical/biocatalytic approach as a new and productive paradigm to upcycling composite polymers not yet touched by modern methods for epoxy recovery.

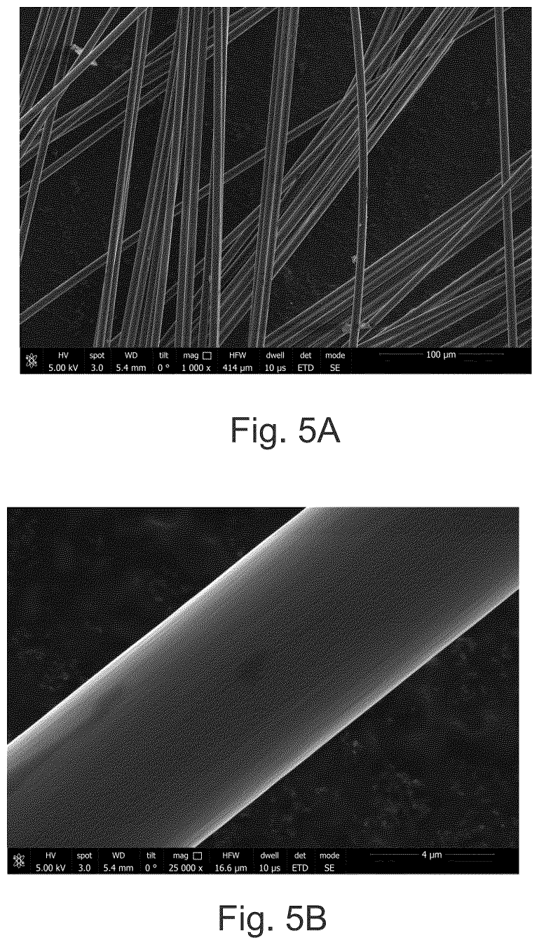

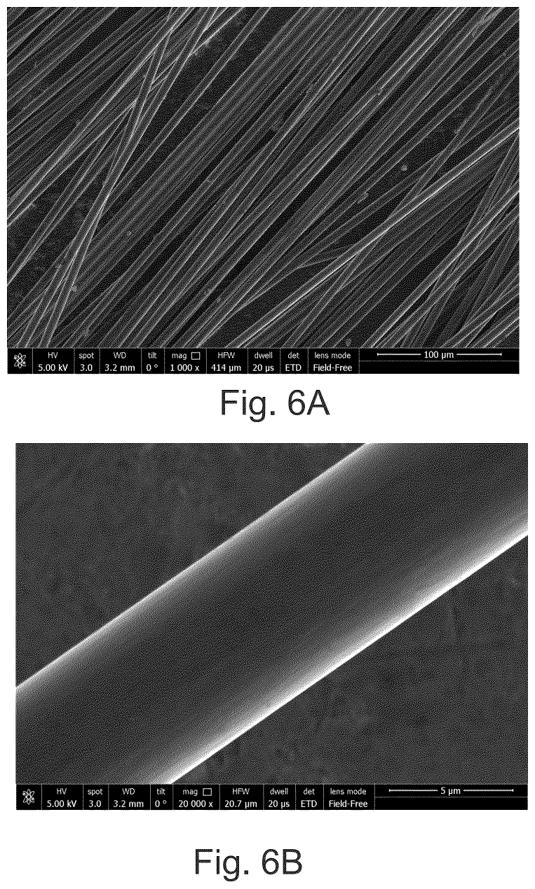

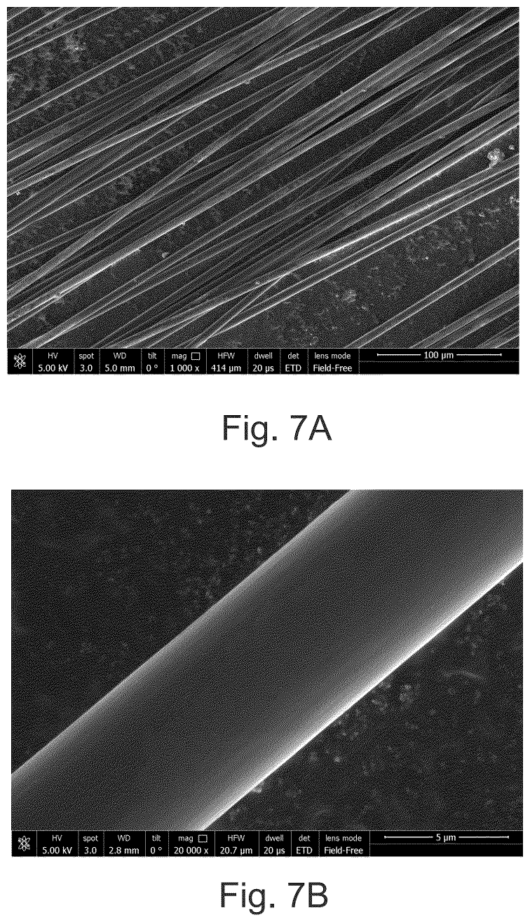

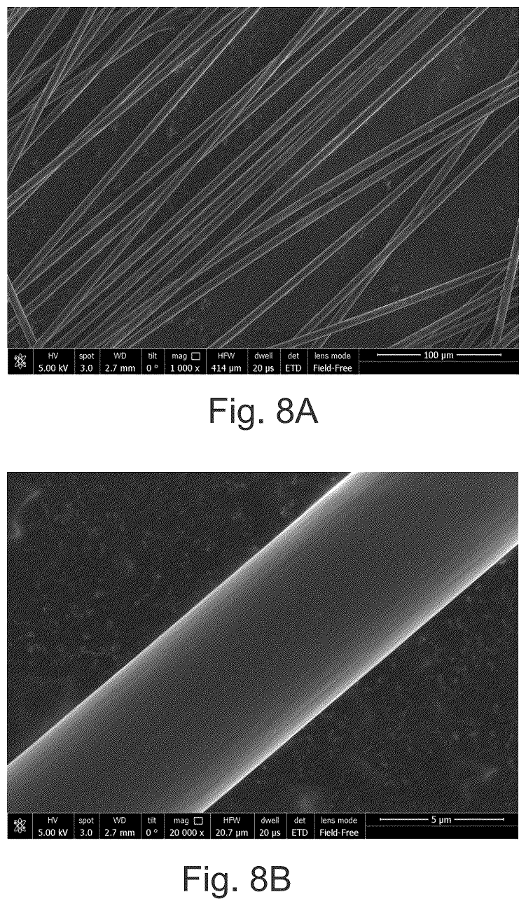

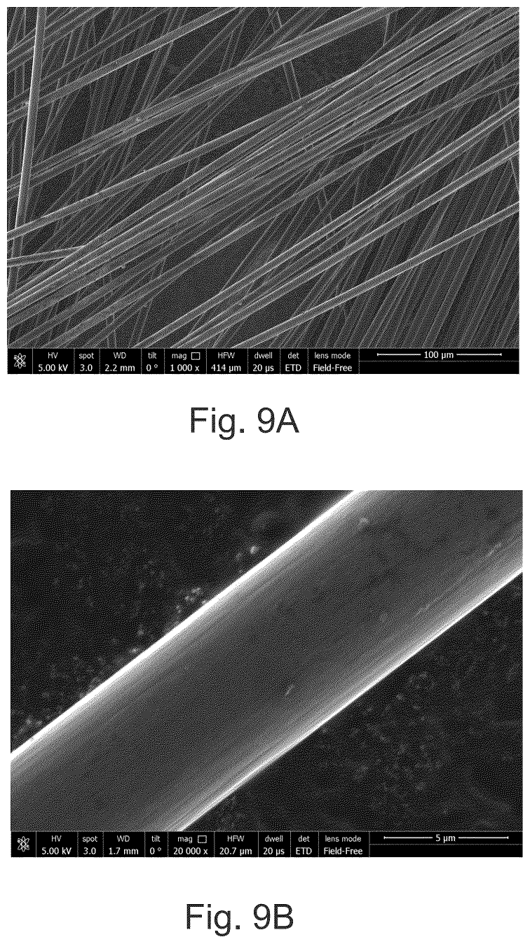

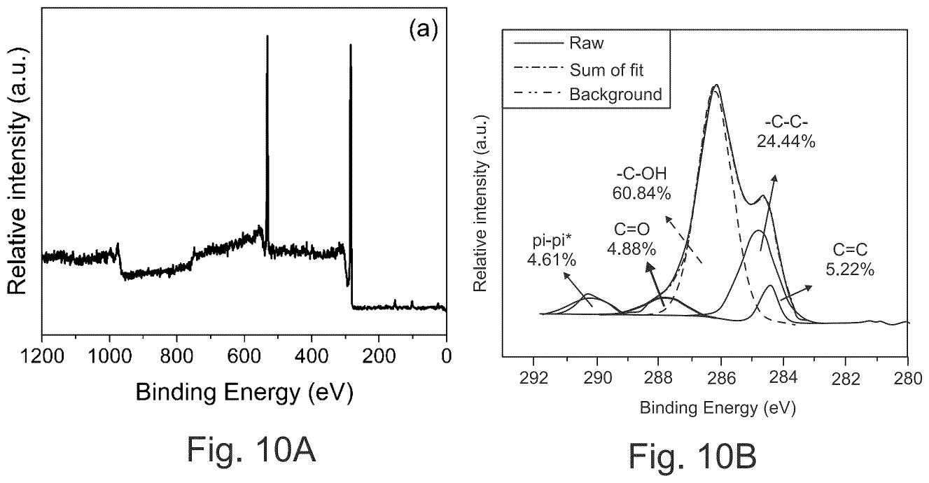

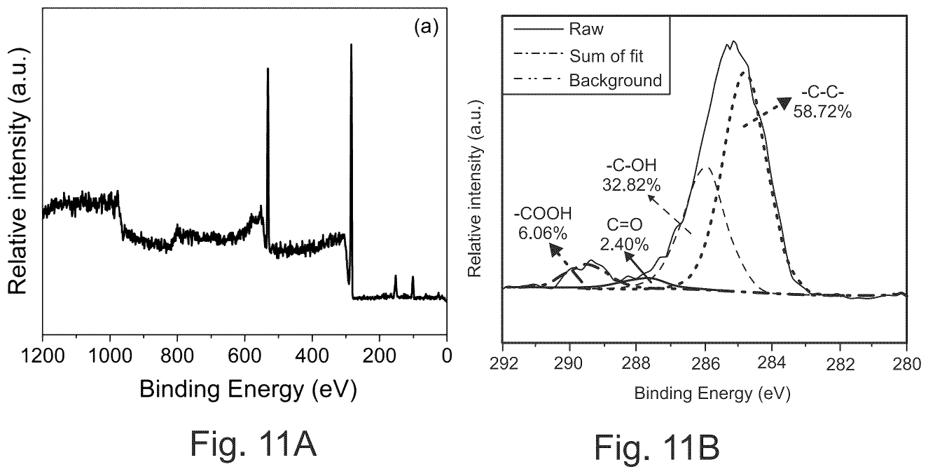

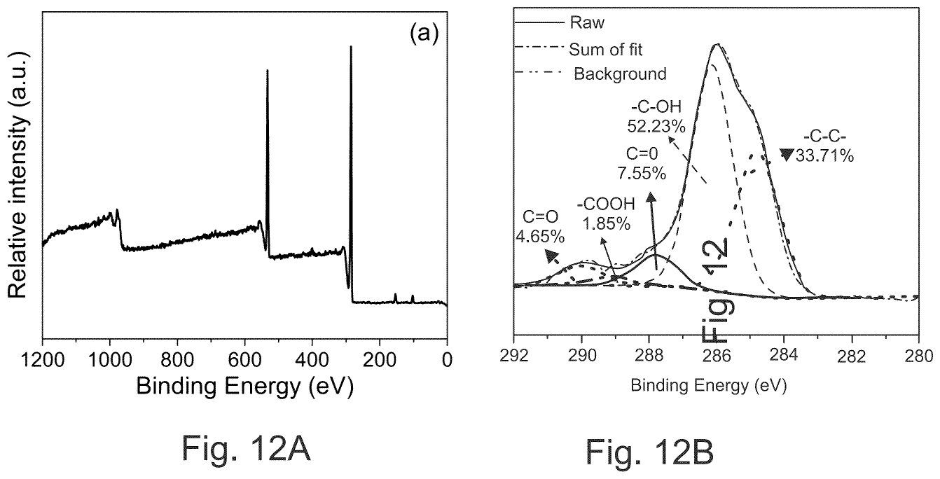

Many reports of composite recycling schemes recover fibers, but few demonstrate their utility as manufacturing substrates. The recovered carbon fibers (rCF) were imaged via scanning electron microscopy (FIGS. 5-9) after being cleaned following the procedure described in Olivar, Clarissa, Yu, Zehan, Miller, Ben, Tangalos, Maria, Jenkinson, Cory B., Nutt, Steven R., Oakley, Berl R., Wang, Clay C. C., Williams, Travis J. “Composite Recycling with Biocatalytic Thermoset Reforming,” Journal of the American Chemical Society 2024, 146 (44), 30004-30008. DOI: 10.1021/jacs.4c10838 and the associated supporting information; the entire disclosure of which is hereby incorporated by reference. These rCFs appeared clean and free of surface damage. X-ray photoelectron spectroscopy (XPS) reported the change in the surface chemistry of the fibers after recycling (FIGS. 10-12 and Table 7). XPS data shows that sizing functionalities, such as hydroxyl and carboxyl groups, diminish but do not disappear during recycling. Diminished sizing is restored by employing a nitric acid treatment, as shown in FIG. 12.

TABLE 7
XPS analysis summary of vCF: virgin CFs; rCF: recovered
CFs; and nrCF: nitric acid treated recovered CFs.

| Surface Element (%) | | | | | | C1s Components (%) | | | | | |
|---|---|---|---|---|---|---|---|---|---|---|---|
| Sample | C | N | Si | O | O/C | C—C | C—OH | C═O | O—C═O | C═C | π-π |
| vCF* | 78.86 | 1.25 | 3.35 | 16.55 | 20.99 | 24.44 | 60.84 | 4.88 | N/A | 5.22 | 4.61 |
| rCF | 74.53 | N/A | 8.17 | 17.3 | 23.21 | 58.72 | 32.82 | 2.4 | 6.06 | N/A | N/A |
| nrCF | 77.54 | 0.32 | 2.79 | 19.35 | 24.95 | 33.71 | 52.23 | 7.55 | 1.85 | N/A | 4.65 |

*with sizing.

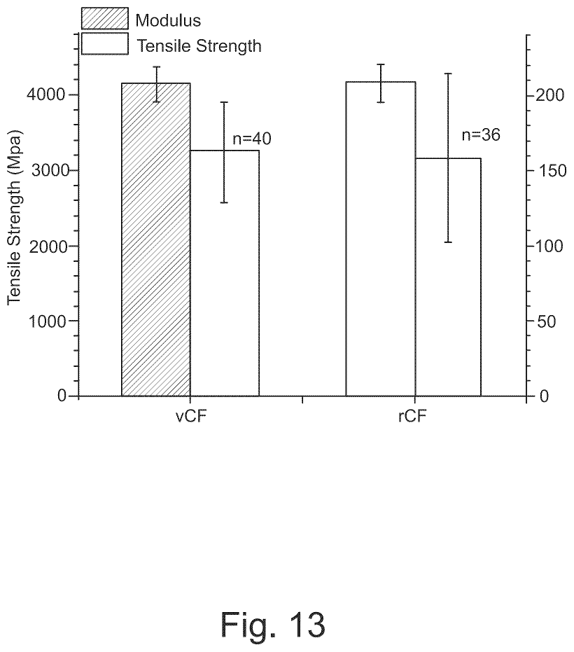

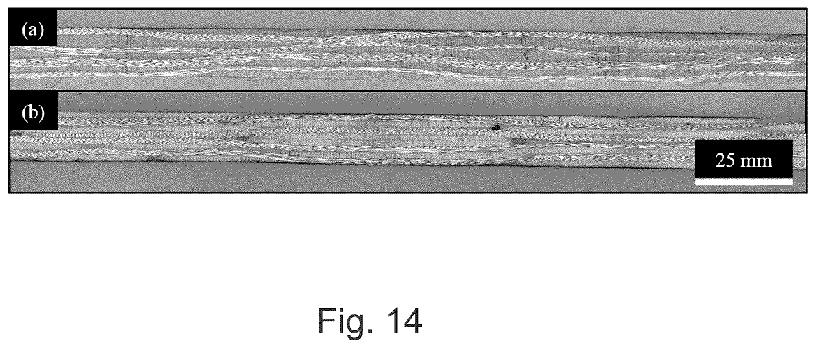

Single-fiber tensile strengths of virgin and recovered CFs were measured and summarized in FIG. 13 and Table 8. It is found that rCFs retained >97% of the tensile strength and >99% of the modulus of virgin fibers. Cleaned but not resized rCFs are readily remanufactured into a second-generation composite with aerospace-grade resin films, cured through a vacuum bag only (VBO, non-autoclave) process. Cross sections of the second-generation composite were polished and examined by light microscopy (FIG. 14). The second-generation composite is well consolidated, demonstrating the viability of upcycling intact carbon fiber fabrics under the proposed conditions.

TABLE 8
Single fiber tensile test summary of virgin CFs (vCFs) and recovered CFs (rCFs).

| | | Strain | Tensile | |
|---|---|---|---|---|
| | Diameter | at Break | Strength | Modulus |
| Sample | (μm) | (%) | (MPa) | (Gpa) |
| vCFs | 7.47 ± 0.13 | 1.49 ± 0.30 | 3244.96 ± 676.10 | 207.28 ± 11.44 |
| rCFs | 7.46 ± 0.12 | 1.48 ± 0.48 | 3145.46 ± 1128.41 | 207.88 ± 12.55 |

The production of both OTA and a second-generation composite coupon from the same starting CFRP sample illustrates the first case of remanufacturing both the CFRP polymer matrix and reinforcing fibers, the latter without disruption of alignment or pattern. While minimal disruption of sizing is observed by XPS, the fabric is remanufactured directly or readily resized with nitric acid. Thus, the first method that fully recovers value from both fiber and matrix components of pre and post-consumer polystyrene containing CFRPs is provided. The procedure is not only rapid, occurring within one week, but also results in high yields of OTA, 172-185% more than previous reports utilizing bacteria instead of fungi. Recovered carbon fibers are readily used to produce a second-generation composite with negligible loss of material properties and minimal, reversible changes to the fibers' surface chemistry.

### 1. General Information

### Materials

Acetone, chloroform, ethyl acetate, dimethyl sulfoxide, n-hexane, methanol, hydrogen peroxide, and D-lactose monohydrate were purchased commercially from VWR. Benzyl alcohol and N-hydroxyphthalimide (NHPI) were purchased commercially from Ambeed. Acetic acid, hydrochloric acid, sulfuric acid, nitric acid, and benzoic acid were purchased commercially from Sigma Aldrich. Tripotassium phosphate trihydrate was purchased commercially from Oakwood Chemical. Manganese nitrate tetrahydrate was purchased commercially from Beantown Chemical. Cobalt nitrate hexahydrate was purchased commercially from Thermo Fisher Scientific. Acetic acid-d₄ and methanol-d₄ were purchased from Cambridge Isotopes Laboratories. D-glucose was purchased commercially from Criterion. All chemicals were used as received, without any further purification.

### Characterization Methods

¹H and ¹³C{¹H} NMR spectra were obtained by a Varian VNMRS 400 or 600 spectrometer at room temperature and processed via Mestrelab Mnova. All the chemical shifts are shown in units of ppm. ¹H NMR data recorded in acetic acid-d₄ and methanol-d₄ are referenced to residual internal CH₃COOD (δ 2.04) and CH₃OD (δ 3.31), respectively. ¹³C{¹H} NMR data recorded in methanol-d₄ is referenced to residual internal CH₃OD (δ 49.15).

HPLC-DAD (analytical) spectra for OTA quantification were acquired using an Agilent 1260 Infinity II mass spectrometer equipped with a reverse phase C18 column (Phenomenex kinetex EVO C18 Column; particle size, 5 μm; column, 4.6 mm×150 mm) with a flow rate of 1 mL min-1. The solvents used were 100% double distilled water (solvent A) and 100% acetonitrile (solvent B), each supplemented with 0.05% trifluoroacetic acid. The solvent gradient used was 0% to 60% solvent B from 0 to 23 min, 60% to 100% solvent B from 23 to 24 min, 100% solvent B from 24 to 25 min, 100% to 0% solvent B from 25 to 26 min, and re-equilibration with 0% solvent B from 26 to 35 min. The OTA peak occurred at 16.8 minutes. A UV lamp of wavelength 300 nm was used, with peak width >0.1 min, 2 s response time, 2.5 Hz.

Preparative HPLC-DAD for OTA purification was completed using an Agilent 1260 Infinity II mass spectrometer equipped with a reverse phase C18 column (COSMOSIL Packed Column 5C18-AR-II; particle size, 5 μm; column, 1.0 mm ID×250 mm) with a flow rate of 8 mL min-1. The solvents used were 100% double distilled water (solvent A) and 100% acetonitrile (solvent B), each supplemented with 0.05% trifluoroacetic acid. The solvent gradient used was: 34% solvent B from 0 to 42 min, 34% to 100% solvent B from 42 to 43 min, 100% solvent B from 43 to 58 min, 100% to 34% solvent B from 58 to 59 min, and re-equilibration with 34% solvent B from 59 to 74 min. The OTA peak occurred between 30-35 minutes.

### 2. Media Recipes and Fungal Metabolism

### Media Recipes

All media were based on minimal medium (MM): 12.0 g/L NaNO3, 3.04 g/L KH2PO4, 1.04 g/L KCl, 1.04 g/L MgSO4·7H2O, 1 ml/L 5.5 M KOH, and 1 ml/L of Hutner's trace elements solution.1 Glucose minimal medium (GMM) contains MM with the addition of 10 g/L of Dglucose. Lactose minimal medium (LMM) is MM with the addition of 15 g/L of D-lactose. Benzoic acid minimal medium (commercial BAMM) contains MM and 10 g/L of commercially available benzoic acid. Pre- and post-consumer composite minimal media, pre-consumer BAMM and post-consumer BAMM, respectively, contain MM and 10 g/L of benzoic acid isolated from their respective composite digests. After adding the benzoic acid substrate, the commercial BAMM, pre-consumer BAMM, and post-consumer BAMM media were adjusted to pH 8 using additional 5.5 M KOH.

### Construction of LO11055

Our starting strain was LO7195 which carries pyrG89, riboB2, and pyroA4 along with a deletion of nkuA and deletions of the following biosynthetic gene clusters (BGCs): sterigmatocystin BGC [genes AN7804-AN7825 using the AspGD, FungiDB gene designations (FungiDB.org) or stcA-stcW plus aflR using the gene symbols assigned by Brown et al., 22] the emericellamide BGC (AN2545-AN2549; easA-easD)3 and the asperfuranone BGC (AN1029-AN1036; afoA-afoG).4 In a co-transformation, we replaced the promoter of AN11191 (alnA) with the promoter of afoE (AN1034) using AfriboB as a selectable marker, and we replaced the promoter of AN11199 (alnB) with the promoter of afoG (AN1036) using AfpyroA as a selectable marker. The resulting strain was designated LO8952. Next, we replaced the wA locus with the gpdA promoter driving the afoA (AN1029) coding sequence using AtpyrG as a selectable marker, creating strain LO8993. In this strain we replaced the yA locus with the afoE promoter driving afoA using the pyrithiamine resistance gene, ptrA, as a selectable marker This strain was designated LO9955. LO9955 has no remaining selectable markers, so we deleted the AtpyrG gene at the wA locus by transforming with a fragment that contained wA flanking DNA as well as the gpdA promoter and the first kb of the afoA coding sequence. Integration of the transforming fragment by homologous recombination evicted the AtpyrG gene resulting in pyr-transformants that were selected on 5-fluoroorotic acid. We designated the resulting strain LO10441. Finally, we replaced the gpdA promoter driving afoA at the wA locus with a 1643 bp sequence containing the AN11489 (metallothionein, mtnA) promoter using AtpyrG as a selectable marker. The resulting strain was designated LO11055. All transforming constructs were created by fusion PCR5 and all strains were confirmed by extensive diagnostic PCR.

In LO11055 the mtnA promoter drives strong constitutive expression of afoA mRNA which is translated into the transcription factor AfoA. AfoA drives expression of alnA and alnB resulting in OTA production. AfoA also drives expression of the copy of the afoA coding sequence that is under control of the afoE promoter resulting in additional production of AfoA. The resulting high levels of AfoA drive additional expression of alnA and alnB, resulting in strong constitutive OTA production.

Sequence of the AN11489 (mtnA) promoter (1646
bases including the ATG start codon for afoA)
(SEQ ID NO: 1)
CTTCAATTCTCAACAGTAGACCCGTTAGTTCCTTCTCGGTGTATACTCG
GCGCACGACTCGGTGGTCTGATCCCACTTGTTTATCAATCAGCGCCAAA
CAAAGCAGTAGCCACCGCTCTAATCCGCATTAATCCTCCAAGACTTCTC
CCACGCAGTAGGCTAGTCTATCAGTTAGTCAGATCCATCATCGTATCTC
CGATCATCGCTTGCGGCTGTCGCGGGTTTCCAAGAGCAGAGCAGTGGGC
ACTGCGCCATTATCGCTAATGCAGGAAATACTTCATAGCTCCATCCTAC
CCCACAATGGCCAGGGCACCAGTTGCACCTGAAACTTCATTGTGGTGCC
CAGGACCCTTCTTGGGCTCTGAGGCATTTTCGCCTTTCGCATTTGAGTA
TGGATACCGATGATACCGCCAAGACAAGAGTCTGTGAGCTGCGCGGGAT
GATAAGGCTTTGAGAAACAGGTCGCAAAGATGGAAATGCCTGAGCTCAA
GCTTCCGAGATCGACTCCAGATGACCGTGTCCGGTGCTGGTCATGGCAA
GGCATTCTCACCAATAGTATCAATCCAGGAAACTACTCTGTTCAAGGAC
TGGAGATAAGATGGTGCCCCGTGCAGCAACAAAGCATACGGGGCGACGT
TAGAGCGCTTGCTTACCTTGCTCAGGACTCCCAACCAAACACCGAATGC
AACTAGCAGTGCTCCCCACTTAAGTAATAGGATATCGTAAATACTGGGT
CGCAAGAAACCATCTTCTTATTGCTAGCCTTACTTTCGGGCTTTACGTA
TGGGGCTTTGAACTATGCAGTATGGAGGAACGTGGCATGACATGGACCT
ATACAGCATTCGGGCTTCCTCTGCATGAAGATCGGAGGAGAGCTTGGTC
TCTTTCGACGGAAAGGAAGATCTTGCTGCTTTCCTCTTGATTCAGAATC
GTTTAACCTGGACATCATATCGCATCTGCATGCAATAACCCAGGCACCT
GAACCCCCACATCTTACCACGCCGAGGCATTCATACAACCTTCCAGCAG
TGAGCCCCTCCCTCCTGGACTACACCAACAGTGATATAGACACGGCGTT
AAGTTTCAGCATGACCTGGTTTTGGCTTGATCGGCAAATCGTAGCAGGG
CGTCAAAGAAAACAAATCGGATGGTAACTGGGGCCTTGCTGTTGTTGCT
ATTGGGGGACGTCTTGCCCGCCAACACACCACCCACACCTGCTGCACCA
CATGGATTCTTGCAGACTGTTCGGTGTCCACTATGCTCCACTGCCAGAC
GCAGTATGCTGAGCCTGTCCAGTCAGAGCACCAAGGGTAAGCTGCGTAG
GTCGTAACCGTGCATGTGCAGGCTGCAATATCGGCGGTTGTAGGCGTGG
GCGTGGGCGTGGGCGTGGGTGAAAGAAGGCTGCCATACTGAGGTGAAGT
CAGTGTCAGATGTAACAGGCGTACTCTTAGTGAGGGGAACTAGTGTAAG
GTAGCAATCCTAGTCCAGGATAGCCAGGAAGAAGAGATATATAAGGAGC
CTCGTCCCCAAGGTTCTCTTCTCTTCCCTATCCATCTGCATCCTATCGC
GACTTCCTCATCCAATCAATCAACCAACCAACCAACCATCTTCCACCGG
TTTATCTACCAACAAACACCATCAACATG

### Culturing LO11055 and Generating a (2Z,4Z,6E)-Octa-2,4,6-Trienoic Acid (OTA) Standard

In four 1 L Erlenmeyer flasks, 2.5×108 spores were inoculated into 250 mL of GMM. The cultures were incubated in the dark for 5 days at 37° C. with shaking of 150 rpm. After the 5-day incubation, cultures were removed from the incubator and filtered in vacuo to separate the mycelia from the media. The media were placed on ice and the pH was adjusted to ca. 3 using 6 M HCl. The media were then extracted three times with equal portions of ethyl acetate (1 L) and the solvent was subsequently dried in vacuo to reveal a light brown solid. This crude media extract underwent preparative HPLC-DAD according to the aforementioned conditions to isolate OTA. The purified HPLC fractions were dried in vacuo resulting in a white crystalline solid. The dry OTA sample was confirmed via ¹H NMR, ¹³C NMR, high resolution mass spectrometry analysis (ESI/QTOF), m/z=137.061 ([M]− calc for C₈H₁₀O₂=138.068). This purified OTA was used to prepare the standard curve for quantification. The sample was stored in the dark at 4° C. to prevent degradation.

### Culturing LO11055 for (2Z,4Z,6E)-Octa-2,4,6-Trienoic Acid (OTA) Yield Comparison

In a 25 mL Erlenmeyer flask, 1.0×107 spores were inoculated into 10 mL of either MM, GMM, LMM, commercial BAMM, pre-consumer BAMM, or post-consumer BAMM. Each condition was completed in triplicate for proper statistical analysis. The cultures were incubated in the dark for 5 days at 37° C. with shaking at 150 rpm.

### Extraction and Quantification of (2Z,4Z,6E)-Octa-2,4,6-Trienoic Acid (OTA)

After the 5-day incubation, cultures were removed from the incubator and gravity filtered to separate the mycelia from the media. For each respective culture, the medium was placed in a 50 mL falcon tube on ice and the pH was adjusted to ca. 3 using 6 M HCl. The media were extracted three times with equal portions of ethyl acetate to media (10 mL) and the solvent was subsequently dried (TurboVap LV). To retrieve any intracellular OTA, mycelia were simultaneously lysed and extracted by vortexing the mycelia in 10 mL of ethyl acetate in a 15 mL falcon tube. The mycelia were extracted once, gravity filtered to remove the hyphal mass, and the resulting extract was dried (TurboVap LV). Medium and mycelia extracts were combined for each respective culture. Samples were redissolved in 10 mL methanol and the yield was determined via analytical HPLCDAD (R2=0.9985) according to the aforementioned method with a sample injection volume of 10 μL. Linear regression was completed using Microsoft Excel ver. 16.87, and modeling of data and statistical analysis of data was completed using MATLAB ver. 2024a.

### 3. Synthetic Procedure

### General Procedure for the Pretreatment of Pre- and Post-Consumer Carbon Fiber Reinforced Polymers (CFRPs)

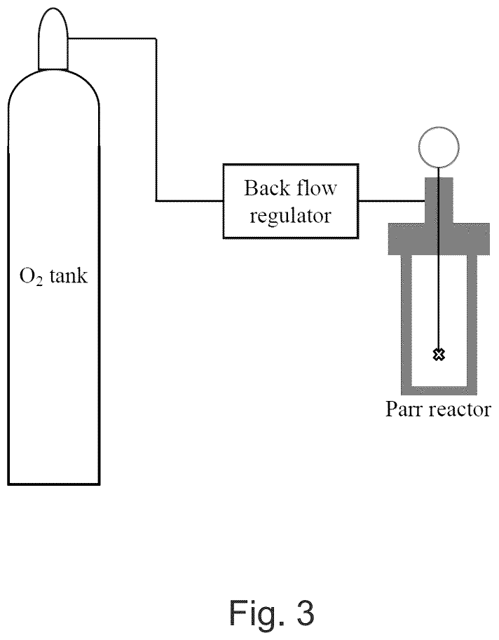

CFRP (38.1×25.4 mm, 4.0981 g, FiberGlast Part #78—Laminating Resin Aropol™ L 67355 T-20 TS, FiberGlast Part #77 Polyester Molding Resin, or post-consumer), benzyl alcohol (100 mL), and tripotassium phosphate trihydrate (0.540 g, 1.98 mmol) were placed in a 500 mL four neck reaction kettle round bottom flask equipped with a reflux condenser and stir bar and heated to 180° C. for 24 hours under nitrogen. The reaction was removed from heat and allowed to cool to room temperature. The benzyl alcohol solution was filtered in vacuo to collect a heterogeneous solid and a clear yellow filtrate. The filtered solid was washed with acetone and dried in vacuo. The carbon fiber plies were washed with acetone, dried with a paper towel, and residual solvent was allowed to evaporate in air overnight. (FIG. 3)

### General Procedure for the Digestion of Pretreated Pre-Consumer (Fiber Glast Part #77 Polyester Molding Resin) CFRPs

The pretreated carbon fiber plies and the resulting solid were added to a 500 mL Parr reactor (FIG. 3) along with manganese nitrate tetrahydrate (0.290 g, 1.16 mmol), cobalt nitrate hexahydrate (0.297 g, 1.02 mmol), N-hydroxyphthalimide (0.294 g, 1.80 mmol), and acetic acid (100 mL). The reactor was closed, sparged with O₂ three times, filled with 3.4 atm O₂, and heated to 150° C. Once the reaction reached 150° C., the pressure on the oxygen tank was increased to 6.1 atm to ensure continuous flow of oxygen into the system. The reaction proceeded for 24 hours while stirring. The reactor was allowed to cool to room temperature before collecting the solution and the carbon fiber plies. The resulting mixture was purified via the procedure outlined below. Isolated benzoic acid: 1.177 g (yield: 93-102%) 1H NMR (acetic acid-d₄, 600 MHz): δ (ppm) 7.49 (t, 2H), 7.63 (t, 1H), 8.09 (d, 2H).

### General Procedure for the Digestion of Pretreated Pre-Consumer (Fiber Glast Part #78-Laminating Resin Aropol™ L 67355 T-20 TS) CFRPs

The pretreated carbon fiber plies and the resulting solid were added to a 500 mL Parr reactor (FIG. 3) along with manganese nitrate tetrahydrate (0.268 g, 1.07 mmol), cobalt nitrate hexahydrate (0.287 g, 0.99 mmol), N-hydroxyphthalimide (0.261 g, 1.60 mmol), and acetic acid (300 mL). The reactor was closed, sparged with O₂ three times, filled with 3.4 atm O₂ and heated to 150° C. Once the reaction reached 150° C., the pressure on the oxygen tank was increased to 6.1 atm to ensure continuous flow of oxygen into the system. The reaction proceeded for 24 hours while stirring. The reactor was allowed to cool to room temperature before collecting the solution and the carbon fiber plies. The mixture was purified via the procedure outlined below. Isolated benzoic acid: 0.401 g (yield: 37-42%).

### General Procedure for the Digestion of Pretreated Post-Consumer CFRPs

The pretreated carbon fiber plies and the resulting solid were added to a 500 mL Parr reactor (FIG. 3) along with manganese nitrate tetrahydrate (0.543 g, 2.16 mmol), cobalt nitrate hexahydrate (0.546 g, 1.88 mmol), N-hydroxyphthalimide (0.556 g, 3.41 mmol), and acetic acid (300 mL). The reactor was closed, sparged with O₂ three times, filled with 3.4 atm O₂ and heated to 150° C. Once the reaction reached 150° C., the pressure on the oxygen tank was increased to 6.1 atm to ensure continuous flow of oxygen into the system. The reaction proceeded for 24 hours while stirring. The reactor was allowed to cool to room temperature before collecting the solution and the carbon fiber plies. The mixture was purified via the procedure outlined below. Isolated benzoic acid: 0.217 g (yield: 16-25%).

### General Procedure for Carbon Fiber Purification

The four obtained carbon fiber plies were washed with acetic acid and ethyl acetate. One of the plies was soaked in diluted HCl (pH 5) for one hour, washed with DI water (150 mL), sonicated in DI water for 5 minutes, rinsed with acetone, and allowed to dry in air. The remaining three plies were sonicated in HPLC grade water for 5 minutes. One of the three plies was washed with acetone and allowed to dry in air. The remaining two plies were soaked in diluted HNO3 of varying pHs (pH 2 and pH 5) for one hour. Both plies were washed with HPLC grade water (150 mL), rinsed with acetone, and allowed to dry in air. The fibers were imaged via SEM (FIGS. 5-9).

### General Procedure for Carbon Fiber Resizing Via Nitric Acid Digestion6,7

Loose carbon fibers recovered after the two-step digestion of pre-consumer CFRPs (FiberGlast Part #77 Polyester Molding Resin) outlined above, were refluxed at 100° C. in nitric acid for 40 minutes resulting in a clear dark orange solution. Carbon fibers were washed with DI water until pH 7. The fibers were soaked for an additional 15 minutes in DI water and the pH of the water was retested to confirm pH 7. Carbon fibers were dried in vacuo at 120° C. for 2 hours and characterized via XPS (FIG. 12).

General Procedure for Benzoic Acid Purification from Pre-Consumer Composite Digest

Solvent was removed from the resulting digest in vacuo resulting in a dark brown product. Ethyl acetate and acidic water (HCl, pH 2) were added to the crude product and filtered in vacuo to remove lose carbon fibers. An extraction was performed with ethyl acetate and acidic water (HCl, pH 2, 3×150 mL). The organic layer was collected, dried over Na2SO4, and decanted. Solvent was removed in vacuo resulting in a yellow powder. Chloroform was added to the yellow powder and the solution was filtered through celite. Filtrate was collected and solvent was removed in vacuo resulting in a yellow powder. n-Hexane was added to the yellow powder, stirred for 30 minutes at room temperature, and the solution was filtered in vacuo resulting in a clear, off-white filtrate and light-yellow powder. Solvent was removed in vacuo affording a shiny off-white solid. Crude benzoic acid was recrystallized in DI water, dissolved in ethyl acetate, and filtered through celite. Solvent was removed in vacuo affording a white powder. The product was confirmed via ¹H NMR.

### 4. Composite Manufacturing and Characterization

### Manufacturing of Pre-Consumer Carbon Fiber Reinforced Polymer (CFRP) Composites

A matrix of 77 and 78 CFRP was formulated using FiberGlast Part #77 Polyester Molding Resin (FiberGlast 77) or FiberGlast Part #78-Laminating Resin Aropol™ L 67355 T-20 TS (FiberGlast 78) with FiberGlast 69 methyl ethyl ketone peroxide hardener. Ninety grams of resin were poured into a mixing cup. The hardener was added to facilitate curing at a ratio of 1% and 1.25% for FiberGlast 77 and FiberGlast 78, respectively. The mixture was stirred for 10 minutes before use. Note that FiberGlast Part #78 was the resumed supply of a substantially similar product following the discontinuation of #77.

A release agent (LOCTITE FREKOTE 770-NC, Henkel) was applied to an aluminum tool plate by wiping it with a cloth. Three coats were applied in total. A layer of pre-cut CF fabrics (203×203 mm, 2×2 twill, FiberGlast 1069) was placed on the coated tool plate. The pre-mixed resin was brushed onto the fabric. After the first layer of fabric was thoroughly wetted, a second layer was plotted on top and compacted by a roller to remove entrapped air. The laminate was built up to four layers of fabric following this procedure. A peel ply and a release film (Airtech, Release Ease 234 TFNP) were placed on the laminate to provide a better surface finish. The laminate was then enclosed in a vacuum bag and compacted by pulling a vacuum.

After lamination, the laminate was cured at room temperature for 24 hours. Fully cured laminates were then cut into 50.8×50.8 mm samples on a water-jet cutter (ProtoMax, OMAX). Resin contents (Wr) for CFRP composites The resin content of CFRPs (Tables 1-3) was determined following the method described in ASTM D3171-22 (procedure B). Fully cured FiberGlast 77, FiberGlast 78, and post-consumer panels were cut into 50.8×10.2 mm specimens on water-jut cutters (ProtoMax, OMAX). Specimens were cleaned with DI water and dried in a convection oven at 80° C. overnight. Each CFRP was weighed to the nearest thousandths place (MI) and placed into individual 250 mL two neck round bottom flasks containing 50 mL of sulfuric acid. The flask was equipped with a reflux condenser and heated to 100° C. in an oil bath. A solution of 30% hydrogen peroxide was added to the mixture in 10 mL aliquots after two, three, and four hours (30 mL total). The reaction was terminated after five hours and allowed to cool to room temperature. Carbon fibers (CFs) were filtered in vacuo and washed with DI water and acetone. Recovered CFs were dried in a convection oven at 120° C. for 12 hours and were subsequently allowed to cool to room temperature. Recovered CFs were weighed to the nearest thousandths place (Mf). The resin content was determined using the following equation:

$${W}_{r} = ( \frac{{M}_{f}}{{M}_{I}} ) \times 100$$

### X-Ray Photoelectron Spectroscopy

X-ray photoelectron spectroscopy (XPS, Kratos Axis Ultra DLD) was used to analyze elements and functional groups on the carbon fiber surface. A survey scan (0-1200 ev) was first acquired on each sample, followed by a high-resolution C1s scan. X-ray source was mono aluminum with a 90° C. incident angle. Curve-fitting of the C1s spectra was performed in CasaXPS software using Shirley baseline and Gaussian-Lorentzian functions, as shown in FIGS. 10-12.

XPS survey spectra show four peaks assigned to C, O, N, and Si. The detailed element compositions of virgin and recovered CFs are summarized in Table S7 (see Exhibit A). The C1s high-resolution spectra can be curve-fitted into five peaks: C—C (284.8 ev), C—OH (286.2 ev), C═O (287.8 ev), O—C═O (288.8 ev) and π-π* satellite (290.2 ev).8 After recycling, C—OH concentration greatly decreased. This indicates that some of the sizing agents of the CFs were removed during hydroxide degradation. The loss of sizing functionality can be restored by treating recovered CFs in nitric acid as shown in FIG. 12.^{6,7}

### Tensile Strength of Recovered Pre-Consumer Carbon Fibers

The tensile properties of both virgin and recovered CFs were tested in accordance with the ISO 11566 standard. Individual fibers were separated from tows and mounted on paper strips. Paper strips were printed and cut into 81.3×25.4 mm, with a 25.4×12.7 mm window in the center. Fibers were mount across the center window. Both ends of the fiber were affixed using double sided tape and epoxy adhesive (Henkel E-20HP) to secure the fibers in place. Once the epoxy adhesive had cured, the mounted samples were examined using a light microscope (Keyence VHX-5000) equipped with a 1500× lens to measure the fiber diameter. The reference length of pixel is calibrated using a reference scale (Keyence OP-87426). The diameter of each sample was measured three times, and the measured values were averaged to obtain the final measurement. The CF cross-sections were assumed to be circular, and the areas were calculated using the averaged diameters.

After mounting the sample onto the load frame, a cut was made in the center of the mounting sheet to free the fiber for testing. The samples were tested at 2 mm/min crosshead speed till break. A total of seventy-six tests were conducted, with forty tests performed on virgin carbon fibers and thirty-six tests on recovered CFs, rCFs. The tensile strength was then calculated and plotted against strain (%) using these calculated areas. The slope of stress-strain curves was taken as tensile modulus. Results are summarized in FIG. 13.

### Manufacturing of a Second-Generation Composite

Cleaned recovered carbon fiber plies (ca. 50.8×50.8 mm) were combined with commercial aerospace-grade resin film, Solvay CYCOM 5320-1, to fabricate prepreg (FIG. 14). Resin films were cut into 50.8×50.8 mm. One layer of resin film was attached to each side of the recovered fabrics. The stack was then heated and pressed on a hot press (Wabash) at 60° C. with 0.1 tons force for two minutes. After cooling down, release films of prepreg were peeled off, and two additional layers of resin film were attached to the prepreg. The stack was then reheated and repressed on a hot press at 60° C. with 0.1 tons force for an additional two minutes. Second generation CFRP panels were laminated via the vacuum bag only process using four layers of prepreg. The curing cycle was as follows (1) hold temperature at 60° C. for 2 hours; (2) increase temperature by 1° C./min until 120° C. is reached; (3) hold temperature at 120° C. for 2 hours; (4) increase temperature by 1.7° C./min until 177° C. is reached; and (5) hold temperature at 177° C. for 2 hours. A reference 5320-1 CFRP was fabricated following the procedure using virgin CF fabrics (50.8×38.1 mm, FiberGlast 1069).

### Second Generation CFRP Cross Section

Strips were cut from the second generation CFRP and the reference 5320-1 CFRP using a water-jet cutter (ProtoMax, OMAX). Strips were mounted with transparent resin (CitoPress-1, Struers) for cross-section polishing. Mounted samples were then polished using silicon carbide papers and aluminum oxide slurry. Polished cross-sections were imaged using a light microscope (200×, VHX5000, Kenyence), as shown in FIG. 14.

Additional details of the present disclosure are found in Olivar, Clarissa, Yu, Zehan, Miller, Ben, Tangalos, Maria, Jenkinson, Cory B., Nutt, Steven R., Oakley, Berl R., Wang, Clay C. C., Williams, Travis J. “Composite Recycling with Biocatalytic Thermoset Reforming,” Journal of the American Chemical Society 2024, 146 (44), 30004-30008. DOI: 10.1021/jacs.4c10838 and the associated supporting information; the entire disclosure of which is hereby incorporated by reference.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. Liu, T., Shao, L., Zhao, B., Chang, Y. C. & Zhang, J. Progress in Chemical Recycling of Carbon Fiber Reinforced Epoxy Composites. Macromol. Rapid Commun. 2022, 43, 2200538.
2. Ma, Y. & Nutt, S. Chemical treatment for recycling of amine/epoxy composites at atmospheric pressure. Polym. Degrad. Stab. 2018, 153, 307-317.
3. Kumar, S. & Krishnan, S. Recycling of carbon fiber with epoxy composites by chemical recycling for future perspective: a review. Chem. Pap. 2020, 74, 3785-3807.
4. Wu, Z. et al. Interfacially reinforced unsaturated polyester carbon fiber composites with a vinyl ester-carbon nanotubes sizing agent. Compos. Sci. Technol. 2018, 164, 195-203.
5. Jiang, D. et al. Reinforced unsaturated polyester composites by chemically grafting amino-POSS onto carbon fibers with active double spiral structural spiralphosphodicholor. Compos. Sci. Technol. 2014, 100, 158-165.
6. Wonderly, C., Grenestedt, J., Fernlund, G. & Cepus, E. Comparison of mechanical properties of glass fiber/vinyl ester and carbon fiber/vinyl ester composites. Compos. Part B 2005, 36, 417-426.
7. Mohsen, S. et al. Fire-retardant unsaturated polyester thermosets: The state-of-the-art, challenges and opportunities. Chem. Eng. J. 2022, 430, 132785.
8. Wu, Y., Fei, M., Qiu, R., Liu, W. & Qui, J. A Review on Styrene Substitutes in Thermosets and Their Composites. Polymers (Basel). 2019, 11, 1815.
9. Navarro, C. A. et al. A structural chemistry look at composites recycling. Mater. Horizons 2020, 7, 2479-2486.
10. Torkaman, N. F., Bremser, W. & Wilhelm, R. Catalytic Recycling of Thermoset Carbon Fiber-Reinforced Polymers. ACS Stain. Chem. Eng. 2024, 12, 7668-7682.
11. Akbar, A. & Liew, K. M. Assessing recycling potential of carbon fiber reinforced plastic waste in production of eco-efficient cement-based materials. J. Clean. Prod. 2020, 274, 123001.
12. Navarro, C. A. et al. Catalytic, aerobic depolymerization of epoxy thermoset composites. Green Chem. 2021, 23, 6356-6360.
13. Cerruti, P. et al. Up-Cycling End-of-Use Materials: Highly Filled Thermoplastic Composites Obtained by Loading Waste Carbon Fiber Composite into Fluidified Recycled Polystyrene. Polym. Compos. 2014, 35, 1621-1628.
14. Chakraborty, I. et al. Massive Electrical Conductivity Enhancement of Multilayer Graphene/Polystyrene Composites Using a Nonconductive Filler. ACS Appl. Mater. Interfaces 2014, 6, 16472-16475.
15. Qi, X.-Y. et al. Enhanced Electrical Conductivity in Polystyrene Nanocomposites at Ultra-Low Graphene Content. ACS Appl. Mater. Interfaces 2011, 3, 3130-3133.
16. Sriprom, W., Sirivallop, A., Choodum, A., Limsakul, W. & Wongniramaiku, W. Plastic/Natural Fiber Composite Based on Recycled Expanded Polystyrene Foam Waste. Polymers (Basel). 2022, 14, 2241.
17. Ballner, D. et al. Lignocellulose Nanofiber-Reinforced Polystyrene Produced from Composite Microspheres Obtained in Suspension Polymerization Shows Superior Mechanical Performance. ACS Appl. Mater. Interfaces 2016, 8, 13520-13525.
18. Rabot, C. et al. Polystyrene Upcycling into Fungal Natural Products and a Biocontrol Agent. J. Am. Chem. Soc. 2023, 145, 5222-5230.
19. Lee, D.-S. et al. Asperlin From the Marine-Derived Fungus Aspergillus sp. SF-5044 Exerts Anti-inflammatory Effects Through Heme Oxygenase-1 Expression in Murine Macrophages. J. Pharmacol. Sci. 2011, 116, 283-295.
20. Grau, M. F. et al. Hybrid Transcription Factor Engineering Activates the Silent Secondary Metabolite Gene Cluster for (+)-Asperlin in Aspergillus nidulans. ACS Chem. Biol. 2018, 13, 3193-3205.
21. Messiha, H. L., Payne, K. A. P., Scrutton, N. S. & Leys, D. A Biological Route to Conjugated Alkenes: Microbial Production of Hepta-1,3,5-triene. ACS Synth. Biol. 2021, 10, 228-235.
22. Xiao, C., Zhang, L., Hao, H. & Wang, W. High Selective Oxidation of Benzyl Alcohol to Benzylaldehyde and Benzoic Acid with Surface Oxygen Vacancies on W18O49/Holey Ultrathin g-C3N4 Nanosheets. ACS Stain. Chem. Eng. 2019, 7, 7268-7276.

What is claimed is:

1. A method for recycling fiber-reinforced polymers (FRPs) comprising:
a) providing a fiber-reinforced polymer, the fiber-reinforced polymer including fibers embedded in a polymer matrix;
b) pre-treating the fiber-reinforced polymer at a first temperature by soaking it in a solution comprising benzyl alcohol and a phosphate base;
c) applying a chemical digestion process to cleave the polymer matrix using a mixture of manganese(II) and cobalt(II) nitrate salts; and
d) recovering fibers and polymer degradation products from the polymer matrix;
e) upgrading the polymer degradation products into secondary metabolites by biocatalysis.

2. The method of claim 1, wherein the secondary metabolites include (2Z,4Z,6E)-octa-2,4,6-trienoic acid (OTA).

3. The method of claim 1, wherein benzoic acid is produced in step c).

4. The method of claim 1, wherein the fibers are selected from the group consisting of carbon fibers, glass fibers, aramid fibers, basalt fibers, natural fibers, boron fibers, and alumina fibers.

5. The method of claim 1, wherein the polymer degradation products are subjected to a fungal biocatalytic conversion process to produce the secondary metabolites.

6. The method of claim 5, wherein an Aspergillus nidulans strain is used to upgrade the polymer degradation products into secondary metabolites.

7. The method of claim 6, wherein the Aspergillus nidulans strain is genetically engineered to enhance secondary metabolite production by:
inserting a promoter of AN11489 (mtnA) to constitutively express AN1029 (afoA), whose gene product AfoA is a strong transcription factor;
placing AN11191 (alnA) and AN11199 (alnB) genes, which encode proteins responsible for synthesis of (2Z,4Z,6E)-octa-2,4,6-trienoic acid (OTA), under control of AN1036 (afoG) and AN1034 (afoE) promoters, respectively, wherein AfoA binds to the afoG promoter (afoG(p)) and the afoE promoter (afoE(p)) to drive transcription of alnA and alnB; and
creating a positive feedback loop by inserting a copy of afoA under the control of the afoE promoter, thereby enhancing afoA transcription and promoting OTA biosynthesis.

8. The method of claim 1, wherein the polymer matrix is composed of a thermoplastic polymer or a thermoset polymer.

9. The method of claim 1, wherein the polymer matrix is polymer crosslinked with an unsaturated polyester or vinyl ester.

10. The method of claim 1, wherein the polymer matrix is composed of polystyrene.

11. The method of claim 1, wherein the fibers retain at least 95% of their initial tensile strength after recycling.

12. The method of claim 1, further comprising cleaning the recovered fibers to form cleaned recovered fibers.

13. The method of claim 12, further comprising manufacturing second-generation composites with the cleaned recovered fibers.

14. The method of claim 13, wherein the second-generation composites are manufactured by embedding the recovered fibers into a polymer resin.

15. The method of claim 14, wherein the polymer resin is an aerospace-grade resin.

16. The method of claim 15, wherein the aerospace-grade resin is cured via a vacuum-bag-only process.

17. A method for recycling carbon fiber reinforced polymers (CFRPs), comprising:
a) providing a carbon fiber-reinforced polymer, the carbon fiber reinforced polymers including carbon fibers embedded in a polymer matrix;
b) pre-treating the carbon fiber reinforced polymer at a first temperature by soaking it in a solution comprising benzyl alcohol and a phosphate base;
c) applying a chemical digestion process to cleave the polymer matrix using a mixture of manganese(II) and cobalt(II) nitrate salts; and
d) recovering carbon fibers and polymer degradation products from the polymer matrix;
e) upgrading the polymer degradation products into secondary metabolites by biocatalysis.

18. The method of claim 17, wherein the polymer degradation products are subjected to a fungal biocatalytic conversion process using an Aspergillus nidulans strain to produce the secondary metabolites.

19. The method of claim 18, wherein the Aspergillus nidulans strain used in the fungal biocatalytic conversion process is genetically engineered to enhance secondary metabolite production by:
inserting a promoter of AN11489 (mtnA) to constitutively express AN1029 (afoA), whose gene product AfoA is a strong transcription factor;
placing AN11191 (alnA) and AN11199 (alnB) genes, which encode proteins responsible for synthesis of (2Z,4Z,6E)-octa-2,4,6-trienoic acid (OTA), under control of AN1036 (afoG) and AN1034 (afoE) promoters, respectively, wherein AfoA binds to the afoG promoter (afoG(p)) and the afoE promoter (afoE(p)) to drive transcription of alnA and alnB; and
creating a positive feedback loop by inserting a copy of afoA under the control of the afoE promoter, thereby enhancing afoA transcription and promoting OTA biosynthesis.

20. An Aspergillus nidulans strain formed by:
inserting the promoter of AN11489 (mtnA) to constitutively express AN1029 (afoA), whose gene product AfoA is a strong transcription factor;
placing AN11191 (alnA) and AN11199 (alnB) genes, which encode proteins responsible for synthesis of (2Z,4Z,6E)-octa-2,4,6-trienoic acid (OTA), under control of AN1036 (afoG) and AN1034 (afoE) promoters, respectively, wherein AfoA binds to the afoG promoter (afoG(p)) and the afoE promoter (afoE(p)) to drive the transcription of alnA and alnB; and
creating a positive feedback loop by inserting a copy of afoA under the control of the afoE promoter, thereby enhancing afoA transcription and promoting OTA biosynthesis.


SEQUENCE LISTING (12 sequences)

SEQ ID NO: 1 - DNA, 1646 bp, Aspergillus nidulans
  source 1..1646: mol_type = genomic DNA
cttcaattctcaacagtagacccgttagttccttctcggtgtatactcggcgcacgactcggtggtctgatcccacttgtttatcaatcagcgccaaacaaagcagtagccaccgctctaatccgcattaatcctccaagacttctcccacgcagtaggctagtctatcagttagtcagatccatcatcgtatctccgatcatcgcttgcggctgtcgcgggtttccaagagcagagcagtgggcactgcgccattatcgctaatgcaggaaatacttcatagctccatcctaccccacaatggccagggcaccagttgcacctgaaacttcattgtggtgcccaggacccttcttgggctctgaggcattttcgcctttcgcatttgagtatggataccgatgataccgccaagacaagagtctgtgagctgcgcgggatgataaggctttgagaaacaggtcgcaaagatggaaatgcctgagctcaagcttccgagatcgactccagatgaccgtgtccggtgctggtcatggcaaggcattctcaccaatagtatcaatccaggaaactactctgttcaaggactggagataagatggtgccccgtgcagcaacaaagcatacggggcgacgttagagcgcttgcttaccttgctcaggactcccaaccaaacaccgaatgcaactagcagtgctccccacttaagtaataggatatcgtaaatactgggtcgcaagaaaccatcttcttattgctagccttactttcgggctttacgtatggggctttgaactatgcagtatggaggaacgtggcatgacatggacctatacagcattcgggcttcctctgcatgaagatcggaggagagcttggtctctttcgacggaaaggaagatcttgctgctttcctcttgattcagaatcgtttaacctggacatcatatcgcatctgcatgcaataacccaggcacctgaacccccacatcttaccacgccgaggcattcatacaaccttccagcagtgagcccctccctcctggactacaccaacagtgatatagacacggcgttaagtttcagcatgacctggttttggcttgatcggcaaatcgtagcagggcgtcaaagaaaacaaatcggatggtaactggggccttgctgttgttgctattgggggacgtcttgcccgccaacacaccacccacacctgctgcaccacatggattcttgcagactgttcggtgtccactatgctccactgccagacgcagtatgctgagcctgtccagtcagagcaccaagggtaagctgcgtaggtcgtaaccgtgcatgtgcaggctgcaatatcggcggttgtaggcgtgggcgtgggcgtgggcgtgggtgaaagaaggctgccatactgaggtgaagtcagtgtcagatgtaacaggcgtactcttagtgaggggaactagtgtaaggtagcaatcctagtccaggatagccaggaagaagagatatataaggagcctcgtccccaaggttctcttctcttccctatccatctgcatcctatcgcgacttcctcatccaatcaatcaaccaaccaaccaaccatcttccaccggtttatctaccaacaaacaccatcaacatg

SEQ ID NO: 2 - protein, 666 aa, Aspergillus nidulans
MACPTRRGRQQPGFACEECRRRKARCDRVRPKCGFCTENELQCVFVDKRQQRGPIKGQITSMQSQLATLRWQLDRYLRHRPPPSITMAGELDEPPADIQTMLDDFDVQVAALKQDATATTTMSTSTALMPAPAISSKDAAPAGAGLSWPDPTWLDRQWQDVSSTSLVPPSDLTVSSATTLTDPLSFDLLNETPPPPSTTTTTSTTRRDSCTKVMLTDLIRAELDQLYFDRVHAFCPIIHRRRYFARVARDSHTPAQACLQFAMRTLAAAMSAHCHLSEHLYAETKALLETHSQTPATPRDKVPLEHIQAWLLLSHYELLRIGVHQAMLTAGRAFRLVQMARLSELDAGSDRQLSPPSSSPPSSLTLSPSGENAENFVDAEEGRRTFWLAYCFDRLLCLQNEWPLTLQEEMILTRLPSLEHNYQNNLPARTPFLTEAMAQTGQSTMSPFAECIIMATLHGRCMTHRRFYANSNSTASGSEFESGAATRDFCIRQNWLSNAVDRRVQMLQQVSSPAVDSDPMLLFTQTLGYRATMHLSDTVQQVSWRALASSPVDQQLLSPGATMSLSAAAYHQMASHAAGEIVRLAKAVPSLSPFKAHPFLPDTLACAATFLSTGSPDPTGGEGVQHLLRVLSELRDTHSLARDYLQGLSVQTQDEDHRQDTRWYCT

SEQ ID NO: 3 - DNA, 2354 bp, Aspergillus nidulans
  source 1..2354: mol_type = genomic DNA
  intron 199..428
  intron 897..949
  intron 1511..1583
atggcgtgtcccaccagacgaggacgacagcagcccggctttgcatgcgaggagtgtcgccgccgcaaagcgcgctgtgatcgcgtgcgtccgaaatgcgggttctgcactgagaatgagctgcagtgtgtgttcgttgacaagaggcagcagaggggtccgatcaaagggcagatcacctcgatgcagtcgcagctgggtaggtgtttgtcttgtctcattgtatctcgtctcgtctgcgcttttgtgattatggggctgccatgtttccggtccggacacaggcatctgcaaggcccgccgctgtgctcccccgatctgcagggaccaatgcagctggttctggagcttgtgctgtgctgcttccctgtctttccacatggtcgagtcgagcgagctagctaacatgggatgcctcatgctttcagcaacgcttcgatggcagcttgatcgatacctgcgacatcgacctcccccgtccataaccatggccggcgagctcgatgagccaccagcggatatccagacgatgctggatgactttgatgtacaggtcgccgcgctgaagcaggatgccacggcaaccaccacaatgtcgacgtcgacagctctcatgcctgccccagccatctcatctaaagatgctgctcctgctggtgctggtttatcgtggcctgacccaacctggctggatcgccagtggcaggatgtcagcagtaccagcctcgtccctccatcagacctgacagtctcgtcggccactaccctaaccgaccctctcagcttcgaccttttgaacgagactcctcctcctccttctacgacgacaacaacgtcgacgacgaggcgagactcatgtactaaggtcatgttaactgacctcatccgggctgaattgtacactacctaactgatttgtctaccatgacacctgactgacaatgtgcagagaccaactctacttcgaccgggtccacgccttctgccccatcatccaccggcgacggtactttgcgcgggtcgcccgagatagccataccccagcacaggcatgtctgcagttcgccatgcgaacgctcgcagcggcaatgtctgctcactgccatcttagcgagcatctctatgccgagaccaaggccctcttggagacgcacagccagacgcccgccacaccgcgagacaaggtcccgctcgagcacatccaggcctggctgttgttaagccactacgagctgctgcggatcggcgtgcaccaggctatgctcacggctggccgggcctttcgtctcgtgcagatggcacgactgtcagagctggatgccgggtcagatcgacagctctcgccgccgtcttcgtcgccgccgtcttcgctaaccctatctccttcgggggagaatgctgagaacttcgtcgacgccgaagaaggccggcggacgttctggcttgcttattgctttgatcgtttgctttgcttgcagaatgagtggccgttaacgttacaagaagagatggtacgtcgcgcttcttttattctatttacctcagaatttatattcagttattttttattctaaccctgctagatattaacccgcctcccctccctcgaacacaactaccagaacaatctccccgcacgcacgccctttctcactgaagccatggcccagaccgggcagagcacaatgtccccgtttgccgaatgcattatcatggccacccttcacggccgatgtatgacgcaccgccgcttctacgcaaacagcaactcgactgcgtccggctccgagttcgagtctggcgccgcgacgcgagacttctgtatccgccagaattggctgtcgaatgcagtggaccggcgagtccagatgctacagcaggtctcctcgcccgctgttgacagcgacccgatgctgctcttcacgcagacgctcggctaccgcgcgaccatgcacctgagcgataccgtccagcaagtctcctggcgggctctcgccagctcgcccgttgaccagcagctactgagcccgggcgcgacgatgtcgctgtcggccgccgcgtaccaccagatggccagccacgcagccggcgagatcgtccgcctggcgaaggccgtcccctcgctgagtccgttcaaggcgcacccgttcctacccgatacgttggcgtgcgccgccacgttcctctcgacgggcagtcccgatcccacgggcggcgagggggtgcagcatctgctacgagtgttaagcgagctgcgcgatacacacagcctggcgcgggattatttgcaggggttgtcggtgcagacgcaggacgaagatcatagacaggatacgaggtggtattgtacatag

SEQ ID NO: 4 - DNA, 1941 bp, Aspergillus nidulans
  source 1..1941: mol_type = genomic DNA
atggcgtgtcccaccagacgaggacgacagcagcccggctttgcatgcgaggagtgtcgccgccgcaaagcgcgctgtgatcgcgtgcgtccgaaatgcgggttctgcactgagaatgagctgcagtgtgtgttcgttgacaagaggcagcagaggggtccgatcaaagggcagatcacctcgatgcagtcgcagctggtggccggcgagctcgatgagccaccagcggatatccagacgatgctggatgactttgatgtacaggtcgccgcgctgaagcaggatgccacggcaaccaccacaatgtcgacgtcgacagctctcatgcctgccccagccatctcatctaaagatgctgctcctgctggtgctggtttatcgtggcctgacccaacctggctggatcgccagtggcaggatgtcagcagtaccagcctcgtccctccatcagacctgacagtctcgtcggccactaccctaaccgaccctctcagcttcgaccttttgaacgagactcctcctcctccttctacgacgacaacaacgtcgacgacgaggcgagactcatgtactaaggtcatgttaactgacctcatccgggctgaattagaccaactctacttcgaccgggtccacgccttctgccccatcatccaccggcgacggtactttgcgcgggtcgcccgagatagccataccccagcacaggcatgtctgcagttcgccatgcgaacgctcgcagcggcaatgtctgctcactgccatcttagcgagcatctctatgccgagaccaaggccctcttggagacgcacagccagacgcccgccacaccgcgagacaaggtcccgctcgagcacatccaggcctggctgttgttaagccactacgagctgctgcggatcggcgtgcaccaggctatgctcacggctggccgggcctttcgtctcgtgcagatggcacgactgtcagagctggatgccgggtcagatcgacagctctcgccgccgtcttcgtcgccgccgtcttcgctaaccctatctccttcgggggagaatgctgagaacttcgtcgacgccgaagaaggccggcggacgttctggcttgcttattgctttgatcgtttgctttgcttgcagaatgagtggccgttaacgttacaagaagagatgatattaacccgcctcccctccctcgaacacaactaccagaacaatctccccgcacgcacgccctttctcactgaagccatggcccagaccgggcagagcacaatgtccccgtttgccgaatgcattatcatggccacccttcacggccgatgtatgacgcaccgccgcttctacgcaaacagcaactcgactgcgtccggctccgagttcgagtctggcgccgcgacgcgagacttctgtatccgccagaattggctgtcgaatgcagtggaccggcgagtccagatgctacagcaggtctcctcgcccgctgttgacagcgacccgatgctgctcttcacgcagacgctcggctaccgcgcgaccatgcacctgagcgataccgtccagcaagtctcctggcgggctctcgccagctcgcccgttgaccagcagctactgagcccgggcgcgacgatgtcgctgtcggccgccgcgtaccaccagatggccagccacgcagccggcgagatcgtccgcctggcgaaggccgtcccctcgctgagtccgttcaaggcgcacccgttcctacccgatacgttggcgtgcgccgccacgttcctctcgacgggcagtcccgatcccacgggcggcgagggggtgcagcatctgctacgagtgttaagcgagctgcgcgatacacacagcctggcgcgggattatttgcaggggttgtcggtgcagacgcaggacgaagatcatagacaggatacgaggtggtattgtacatag

SEQ ID NO: 5 - DNA, 802 bp, Aspergillus nidulans
  source 1..802: mol_type = genomic DNA
gtctaaaggcgtccactgcatatatatcagccactgtagtcgtgttatgtacttatacacacaaacgtatgtgtatatattgaaaatgtggcattatcaacctctatctggctatgtagcatctatagtagcttgcattttgggaaattcttgggatattgcattggcctccgcaataactatttcaacaggcaatttctgccccatttacctttgatccccagacggcagccatacccagatacaagtccagacaacgcccaatcagcccaaccagccctgcatccatatgctgagttggggtttccctaaaggctctacagaagacgacaaagctgagacgaaaagctgagacaagcatcagccagcggacttccggactagcgaagctgagtctcgatctggagtcttggatggagtcctgtagtccgaaaagtccgccagaagcgccaccaggcttagggcttacagcgctgcagccagaaggattccatcttaccttaatctccggctccaatcagagtgccccaacgatgtcagtgtttcctcagtgcgtggcttcccgcaacatgtgctaaggtagaagtattagccctacttccccaagggacagacatacatcttgtctatctactacagacacccagtctatcatattctctctgctctcttgacccgctcgctcgcctggaaagtcaacctcaagtgtctcaactacacctcattcattcacagttgacccggcagagcacttagcactatcaactacctagctaccctcctaccctcctaccctcccgca

SEQ ID NO: 6 - DNA, 909 bp, Aspergillus nidulans
  source 1..909: mol_type = genomic DNA
gttttcacatgagctacccgttcattataaccatcagctctagctaggacaggatcgcatgagtatatacctatttatattccttccctcccaactcggactcacgctttatatatatgtctactattactcgtgggtgaagagaagtttacgactatttagcctagatgaaggataggttgtgcaatgctcgatagcgtagcatttaaccctacctagtaatgagctacttgggctgctagaataaatctcccaatccaagctaatgtagtcagagctgaacgcaagtctcgtacatggccctacgaggcatcacaatagccctaaagagtatcacgtgaccatactagcaccgcaatgagttcaggatccgacaatagcgaggctgtatccaagtgcgccgaataatgtctatcactgtagaaatatatctgattcgctcagctggtcgataggcgaagcatcggagttggcggagttggcggagttgcaggacttgctggattagggctgaggtcagacggactctcactctccgctatagacactgggcgatgttgtaggcagcgatgggagaatgtgcattgcacatggtccggagatttctggagtcaggtcatgcagtctagatcctgactgcagtagaatgtgcagattccggagcttggggagttaacctgcagtaagctcagctcaagcaatgatcggtaggtaggcctggtggccatatcagctatagatgcgatccgcgcctcaagcgcatttcaagccctccctcttcaatacgtttgcgataccttagagaaacaaatcaacatccatcaactggcacagattcatctaccaactcaacgtgattacccgtccagctttgacctaaacctccataatccccatccacaaggcacc

SEQ ID NO: 7 - DNA, 7700 bp, Aspergillus nidulans
  source 1..7700: mol_type = genomic DNA
  intron 312..383
  intron 528..585
  intron 854..906
  intron 6693..6752
  intron 7434..7518
atgaatctgccgggcgaaattcatcataggtttacttatatgtacattgcgcaggtcagctttgcagacctcgacattaccaaacacaacttaccaatctcaacgatgtcttcttcagattccccgaactatggcgatgccacgatgcccattgccatcgttggcatggcagcccggttctctggtgaagcaactaacccatccaagttatgggatatgatggtacagggtcgaactgggcattccgcagttccagagaatcgtttcgacgcggaggcgtggcatcatcccagccacgaacgaagagggactgtaggtttatgaattcaaaattggcaaaaaaaatggattgatgcaacgtgctaatgatgccatatggctagatccaaccacgaagtggcttcttccttcgtgaggaccctgccgtttttgacgcaccatttttctcgatgacggcgaaggaagctgcaggaatggacccaatgcagagaaaacttttagaaatttcttacgaggcttttgaaaatggtaggctgtccgataagaaatatccgtaaaaacagaaatgctgaactcaaccaacagctggcatcccgattaccaagttgcctggtacggcgaccggcgtctacagtggtgttatgacgaacgactatgagttgatgactgccggtgatccaatgcagttgccacagaatgcagcatcgggaacaagccgtgctatgcttgccaatcggatttcctggttctatgatcttcggggtcccagcttcgcgctcgatacggcttgctcgtccagtttatatgcgctacacctggcttgtcagtcactgcaagcaggagagactgaccaggtacgcatactcactcgctaggaaggaagaccagcgttgacagtgattacaggcgctcgtaacaggagtcaatcttatcctagcgccaaattttatctcccagctttcttcgatgcacatgctgagcccagacgggaagagccattctttcgactctcgtgccaatggctacgccagaggcgaggcccttgctgcagtcgtggtcaaaccactctatcaagcactcgctgacggtgacactatccgagcagttattcgaggaagtggtgcaaatcaagacggtaagaccgttggaatcacaattccaaacccgcaggcgcaagcagaacttatccgcaaaacatacgctacagctgggctgggtctcgaacagacagggtatttcgaggcacacggcacaggaacccctgtaggtgatccaatcgagctgagcgctattgggacaagtttcggcgagcatcgcagccagaactgtccgctttttgtgggaagcgtgaagacgaatgttggccatacggagggggctgccggtctggcaggcgttgtgaagacagtgctagctctggaggcgggtattattcctccactggctgactttcaggagctgaatgaaaagcttcgacttgaggagtggaagttggctctcccgctcaaagcaactccttggccgatgccgggtcttcgccgggcaagtgtcaactcgttcggttttggaggtgccaatgctcatgttatccttgatgatgcctatcattacctgaaatcccacgggctgagcgcaaaccatcatacaacactgtcggaaagcgaggactcatcggattcaggtctggaaatggactcctcaaccagtgacagtggggaagggcagtcgagcaagttgttacttttctcagcgtatgatggcgctggtattaaaaggacagaagcctcgtggaacagccaccttgccgatatcctcgcagacagcaaaaccgtggacgagaccatggggatgaatgacctcgcatacaccctttctgaccgccgcacgaccttcgacttccggagctttgctgtggcatcgagcgtacaagatctgaaagctaagcttgagaacgacggccttccgcgactgaacagggcctctcgtcgctccaaccctgtgtttgtgttcacgggccagggagcccaatggcccgcgatggggcgagaactgctgtctaaccctatctttcgcgccagcatcgagcgtagcaaagccgttctcgaacttgaaggctgcgagtgggatgtggtgcaagttttgtcagatccgcaggaccagcgcatccatatcccagccttttcccaaccagtctgcacaatcttacaggtcgctcttgttgatcttctgcagtcatggggcattcaacctgcagcaacggttggtcattccagtggagaggttgcggcggcttatgcagcgaaaatgatatcccaggacgaggctgtacgaattggatactggcgaggcttctacagtgagcaggtgaaggctcgactagaaaatatacgaggttccatgatggccgtcggtctatcagaatctcaggccacttcgtacctaaaccgggtaccagaaggcagtgtagtcgttgcctgtatcaacagcccgtccagtgtcactttatctggcgaagatcattcgatcaagactctcgaagcaatcctgcaggcagacggccactttgcgcgtaagcttcgtgtggaggttgcctaccattctcctcacatgaagaccgttgcagatgagttcctgaacgctgttggcataatcactccacagccttctgaaatcccgatgttcagttcggttacggaaacccgggttgaggacccagcgactctcgttgcttcatactggatgcagaacctgatatccccagttcgtttctctggtgcactagcaaccctactaaatgacacccccagtgtaaaggcaaatactcgccgtcgacgcactgctggtattgtctggagtgctctgatcgaggttggaccccatgaggcactgaaagggccatgccgtcagatcatgtcgggcctaaacaccaaattagcagaccagattccttatatgtctgtccttagccgcggtaagagtgcagtggagacatcactgacagcagctggcctcctttgggcgtcgggacatccgatcaatatacgtgaggtaaaccagtatcgggatactggtgagagggtgatcactgacctgccaccgtacccctggaaccatgaaaagggcttttggcatgaacctgcggcctctatatccgcacgattgagaaaagaaccacgcaacgatctcctgggtgtgccagtggcgcagcaaaaccctttcgagcggtgctggcaaaactatctctccgtctcagaatgtccttggcagaaagatcacgtcattactggcactgtactgtatccgggagctggacatctgattatggcctttgaagctgccatccggctggctgctgacaatagaccgctgaagggagtctcgttctctgatgtccactttgacaaggggcttgtcatcccgtcggacgaccatggcgtcgagacacgactctgcacacggcctcatgagagcctgttagactggtaccactacactttatactccatcaacgccactggagactggacaaaacactcttggggctcgttcagcctccactacgaggatgctgtcagcgtgcagcaagcgaaacgcagcaaaggcgaatacgacgatatcaacactcgtgcatgtcggaagctggatgttgagtcgttctacgagcagctcctgtcaatcggcacagaatatgggccgacattccgcaacttagtacatgctgctgcggctcctggttaccacagcggcgtgggtaccatcacaataccagacactaaatcagtcatgcctcacgagtttgaatatcctcacttgatccatcccgccacgctggatgccatctttcacttgatattcgtggccatgggcgagggcaacgcgctctccgagtctgccatcccaacacgggtagaccgcatttacatttccacggatctgcctcgaggtgtcggagccaagtatacaggttacggtcgcgcggagcctgtctccagccgcgataccttggggactatcgtcgtctcggatgaaaattggtcagcaggacccaagatcattgttgaagggatgactgtcacggaagtctctgctggtgcttctacctcattcaactccttgcttataccggggggccaaggtcgcattgccacgcttgaatggaaggaggatgtggactcacttgtcgggccgacggctgagtcgtggctggcccagaaagggccaagtattgggggccaggccagcgatgtgactgaagcggtgcagcgcctcgacgcttggctggaactttcctgtttcaagagcacagaccttggcacccttgttatatgtccctcgaaattgaaaggcagttttgaactcgtcaagaaatatggctctaagcatggagaaaggtatcggttcggccggaccacgatcatcgaattttccgagaatgatatttcagcagcagagagtgctttcgcaccgcacggaattgaatcttcatatgctgctatcgatctttcagccactcctgagcatgctatggaacaattggggatgttcgatttgatcatcgctgaggagaatgttattgtccaatttcccgatgtcacaaagatactgcatcgggaaggcagagtcgctattattaggagtcatgctttacccgatgagcgccacttcgcagcaacaaagggcttactgaaggaaatctcatttgagtctcaggacggctctatcctacaaattgctggtttgggattggaaatggatccagccattcgcagccttgatgacgttgtactgttacaacatgtagatgcttccccagctgccaaaaactttgaaaaaaggcttacggcccagctaaccagtctcggtgcgcatgtacggagcaataccatagcaaacgcgagcagcctttcaggaaacatcgtgatctctttgctagagattgattgtcaatttgtcatatcatggacatcggaagaattcgaacaattccgccagctgaccaacgcgagatacgtcctatggattacgcgcgggggcttgctcgacgcagaccgggcatcgcttgattatgcgccgtccaccggccttctgcgtactgtccgcgttgaaaagccccaaattcgactgcctcacttggatctctcccctagcctggacttgaactcggatcgcgcggttgaaattgtgatatctgcattccactccagcatcaagccctctgtaaaggaaaagaatctcgaaatggaatatgcagagtccaacgggctattatacatcccaagagctcgagggcatgcggccttagaccacgagcttgcccttcggggtgagaaagtgtccagcatcccggggccactgtctgcgcctggaatagcacgacgacttgagacttccctggctggaagcccatctcaggctcgctgggttcctgacgagacagtcggagataagctggcagattttgatgttgaaatccaggtctctcacgtgggcttggagcacagtaaggtcgaaaattatctaaatggaaaacagctttcgctagcacctggcctcgggcgcgtggcagttgggaccctgaccagggctggtgcaaaagtttcacgattcattcctggtgaccaagtgtttgctttgcacgccgcacctttccatacacacctgcgcgtcaccgaagacgctgtccacgcagtcccggacatcctgtctccagctcaggctgcacatcttccgctagctgcggctcgagcctggcactcgcttatcgatgttgcagcgttccgcgctggtcaatctgtctttgttaatggtgcaagcgatactgtcgggcgactaactgttgagctggcgcggcttctgaagggggatgtttttgcctctgttagctctgatgacgaaaagcacactctgacgaaaacctacaacataccagaagatcatattttcagcctttcacaccgaactgactgggcatcagacctcaaagccgcgatgggacaggggcagctagatattgtgatcaacaacgcaacgccaagcccagcaattcgcagccttttccaatcaattgcaccaaaaggacgattcgtagaccttacccagcgtctcgatccgtcgttgctcgacccaaggatgttccagcggaacgttactttgtccttggtggattgggagtctttgaccaacccgcagctgggcgccctgatggttagatcactagatttgctacgagcaggggccttaaccccgatcaaggaagaatatatcttttcggtatctgacctgccagaagcattattgtccgtcggccaacagcaacatgagcgagtcgcagcaccggtggtggtggagttctccgcagatgccacagttcctttacttccatctcttcccgcaccacttcaccttaaacccgatgcgacctatattctcgctggggggctgggtgctcttgggcttaccattgcagagaatatgtgtagccacggtgccggccacttggtgttcctcagccgctccggggcatcgagccaaagacagcaagaggcgcttgagagcttccgtgctcgcggatgcaaggtcgacgtggtgaagtgtgacgtgacggaccaagaacaggtgcaagctctggctacccagattcgggagcaatcgtggaatgttcgcggcatcatccaactggcaatggttctccgggtatgtaattggacttcattcaccgtgaccggctaacccactgaccttattgcttgcaggactcgatcttcgaaaatatgaccttcgataagtgggaaacagctgtcaaccctaagatcaaaggaacgtggaacctgcacgccgagcttcccaaggatgttgatttcttcatcattctctcttcgctctctggcatcattggaaatactgcccaagctaactactgcgccggtaatacatatgaggacgctcttgcccattataggcgtaagcagggcctcgccgcgacgaccctcaacgtcggcctggtcacggacgcaagccacttcaacgagaattccacaatcgaagactatctgcgtaaatatagtcactggatagccgcccaggtcaccgacagcgagctgcagcacaccatcactgccgtgatgcggaggacagtcggcgacaagaatgagcccgtacccgaccagctcctggtcggtctctcagataacgtccggcgtgacggaaacagtctgaatttgtggccacaggaccgcaaattcgaccacaggatcagtctagaagatgggctcggtgtggttgagaaggatacgaatcaacagaaattaaaggcatccacgacagtcgctcaggcgcacgaggtcgttgagacagcattacgcttgaatgttgccgcggctatgactgcctccccggatgatattgacattgagaaaccgctctatgcctttggcagtatgtattcatctctctctatccttccgtctcatctcatcacagctccattcgcttcctgcccagttactaacatgtagacagttgactcgcttaaagggattgaagtccgtaattggatttttagcgagctgcaggccgatgtatccgtgtttgaggttcttagtccgatgaccctaagccgcctggctctcaagattgtttcgaagagcacgctcgtgggcgcggagcttgctgcagaagctgcggcggacagtgtggcgtaa

SEQ ID NO: 8 - DNA, 7377 bp, Aspergillus nidulans
  source 1..7377: mol_type = genomic DNA
atgaatctgccgggcgaaattcatcataggtttacttatatgtacattgcgcaggtcagctttgcagacctcgacattaccaaacacaacttaccaatctcaacgatgtcttcttcagattccccgaactatggcgatgccacgatgcccattgccatcgttggcatggcagcccggttctctggtgaagcaactaacccatccaagttatgggatatgatggtacagggtcgaactgggcattccgcagttccagagaatcgtttcgacgcggaggcgtggcatcatcccagccacgaacgaagagggactatccaaccacgaagtggcttcttccttcgtgaggaccctgccgtttttgacgcaccatttttctcgatgacggcgaaggaagctgcaggaatggacccaatgcagagaaaacttttagaaatttcttacgaggcttttgaaaatgctggcatcccgattaccaagttgcctggtacggcgaccggcgtctacagtggtgttatgacgaacgactatgagttgatgactgccggtgatccaatgcagttgccacagaatgcagcatcgggaacaagccgtgctatgcttgccaatcggatttcctggttctatgatcttcggggtcccagcttcgcgctcgatacggcttgctcgtccagtttatatgcgctacacctggcttgtcagtcactgcaagcaggagagactgaccaggcgctcgtaacaggagtcaatcttatcctagcgccaaattttatctcccagctttcttcgatgcacatgctgagcccagacgggaagagccattctttcgactctcgtgccaatggctacgccagaggcgaggcccttgctgcagtcgtggtcaaaccactctatcaagcactcgctgacggtgacactatccgagcagttattcgaggaagtggtgcaaatcaagacggtaagaccgttggaatcacaattccaaacccgcaggcgcaagcagaacttatccgcaaaacatacgctacagctgggctgggtctcgaacagacagggtatttcgaggcacacggcacaggaacccctgtaggtgatccaatcgagctgagcgctattgggacaagtttcggcgagcatcgcagccagaactgtccgctttttgtgggaagcgtgaagacgaatgttggccatacggagggggctgccggtctggcaggcgttgtgaagacagtgctagctctggaggcgggtattattcctccactggctgactttcaggagctgaatgaaaagcttcgacttgaggagtggaagttggctctcccgctcaaagcaactccttggccgatgccgggtcttcgccgggcaagtgtcaactcgttcggttttggaggtgccaatgctcatgttatccttgatgatgcctatcattacctgaaatcccacgggctgagcgcaaaccatcatacaacactgtcggaaagcgaggactcatcggattcaggtctggaaatggactcctcaaccagtgacagtggggaagggcagtcgagcaagttgttacttttctcagcgtatgatggcgctggtattaaaaggacagaagcctcgtggaacagccaccttgccgatatcctcgcagacagcaaaaccgtggacgagaccatggggatgaatgacctcgcatacaccctttctgaccgccgcacgaccttcgacttccggagctttgctgtggcatcgagcgtacaagatctgaaagctaagcttgagaacgacggccttccgcgactgaacagggcctctcgtcgctccaaccctgtgtttgtgttcacgggccagggagcccaatggcccgcgatggggcgagaactgctgtctaaccctatctttcgcgccagcatcgagcgtagcaaagccgttctcgaacttgaaggctgcgagtgggatgtggtgcaagttttgtcagatccgcaggaccagcgcatccatatcccagccttttcccaaccagtctgcacaatcttacaggtcgctcttgttgatcttctgcagtcatggggcattcaacctgcagcaacggttggtcattccagtggagaggttgcggcggcttatgcagcgaaaatgatatcccaggacgaggctgtacgaattggatactggcgaggcttctacagtgagcaggtgaaggctcgactagaaaatatacgaggttccatgatggccgtcggtctatcagaatctcaggccacttcgtacctaaaccgggtaccagaaggcagtgtagtcgttgcctgtatcaacagcccgtccagtgtcactttatctggcgaagatcattcgatcaagactctcgaagcaatcctgcaggcagacggccactttgcgcgtaagcttcgtgtggaggttgcctaccattctcctcacatgaagaccgttgcagatgagttcctgaacgctgttggcataatcactccacagccttctgaaatcccgatgttcagttcggttacggaaacccgggttgaggacccagcgactctcgttgcttcatactggatgcagaacctgatatccccagttcgtttctctggtgcactagcaaccctactaaatgacacccccagtgtaaaggcaaatactcgccgtcgacgcactgctggtattgtctggagtgctctgatcgaggttggaccccatgaggcactgaaagggccatgccgtcagatcatgtcgggcctaaacaccaaattagcagaccagattccttatatgtctgtccttagccgcggtaagagtgcagtggagacatcactgacagcagctggcctcctttgggcgtcgggacatccgatcaatatacgtgaggtaaaccagtatcgggatactggtgagagggtgatcactgacctgccaccgtacccctggaaccatgaaaagggcttttggcatgaacctgcggcctctatatccgcacgattgagaaaagaaccacgcaacgatctcctgggtgtgccagtggcgcagcaaaaccctttcgagcggtgctggcaaaactatctctccgtctcagaatgtccttggcagaaagatcacgtcattactggcactgtactgtatccgggagctggacatctgattatggcctttgaagctgccatccggctggctgctgacaatagaccgctgaagggagtctcgttctctgatgtccactttgacaaggggcttgtcatcccgtcggacgaccatggcgtcgagacacgactctgcacacggcctcatgagagcctgttagactggtaccactacactttatactccatcaacgccactggagactggacaaaacactcttggggctcgttcagcctccactacgaggatgctgtcagcgtgcagcaagcgaaacgcagcaaaggcgaatacgacgatatcaacactcgtgcatgtcggaagctggatgttgagtcgttctacgagcagctcctgtcaatcggcacagaatatgggccgacattccgcaacttagtacatgctgctgcggctcctggttaccacagcggcgtgggtaccatcacaataccagacactaaatcagtcatgcctcacgagtttgaatatcctcacttgatccatcccgccacgctggatgccatctttcacttgatattcgtggccatgggcgagggcaacgcgctctccgagtctgccatcccaacacgggtagaccgcatttacatttccacggatctgcctcgaggtgtcggagccaagtatacaggttacggtcgcgcggagcctgtctccagccgcgataccttggggactatcgtcgtctcggatgaaaattggtcagcaggacccaagatcattgttgaagggatgactgtcacggaagtctctgctggtgcttctacctcattcaactccttgcttataccggggggccaaggtcgcattgccacgcttgaatggaaggaggatgtggactcacttgtcgggccgacggctgagtcgtggctggcccagaaagggccaagtattgggggccaggccagcgatgtgactgaagcggtgcagcgcctcgacgcttggctggaactttcctgtttcaagagcacagaccttggcacccttgttatatgtccctcgaaattgaaaggcagttttgaactcgtcaagaaatatggctctaagcatggagaaaggtatcggttcggccggaccacgatcatcgaattttccgagaatgatatttcagcagcagagagtgctttcgcaccgcacggaattgaatcttcatatgctgctatcgatctttcagccactcctgagcatgctatggaacaattggggatgttcgatttgatcatcgctgaggagaatgttattgtccaatttcccgatgtcacaaagatactgcatcgggaaggcagagtcgctattattaggagtcatgctttacccgatgagcgccacttcgcagcaacaaagggcttactgaaggaaatctcatttgagtctcaggacggctctatcctacaaattgctggtttgggattggaaatggatccagccattcgcagccttgatgacgttgtactgttacaacatgtagatgcttccccagctgccaaaaactttgaaaaaaggcttacggcccagctaaccagtctcggtgcgcatgtacggagcaataccatagcaaacgcgagcagcctttcaggaaacatcgtgatctctttgctagagattgattgtcaatttgtcatatcatggacatcggaagaattcgaacaattccgccagctgaccaacgcgagatacgtcctatggattacgcgcgggggcttgctcgacgcagaccgggcatcgcttgattatgcgccgtccaccggccttctgcgtactgtccgcgttgaaaagccccaaattcgactgcctcacttggatctctcccctagcctggacttgaactcggatcgcgcggttgaaattgtgatatctgcattccactccagcatcaagccctctgtaaaggaaaagaatctcgaaatggaatatgcagagtccaacgggctattatacatcccaagagctcgagggcatgcggccttagaccacgagcttgcccttcggggtgagaaagtgtccagcatcccggggccactgtctgcgcctggaatagcacgacgacttgagacttccctggctggaagcccatctcaggctcgctgggttcctgacgagacagtcggagataagctggcagattttgatgttgaaatccaggtctctcacgtgggcttggagcacagtaaggtcgaaaattatctaaatggaaaacagctttcgctagcacctggcctcgggcgcgtggcagttgggaccctgaccagggctggtgcaaaagtttcacgattcattcctggtgaccaagtgtttgctttgcacgccgcacctttccatacacacctgcgcgtcaccgaagacgctgtccacgcagtcccggacatcctgtctccagctcaggctgcacatcttccgctagctgcggctcgagcctggcactcgcttatcgatgttgcagcgttccgcgctggtcaatctgtctttgttaatggtgcaagcgatactgtcgggcgactaactgttgagctggcgcggcttctgaagggggatgtttttgcctctgttagctctgatgacgaaaagcacactctgacgaaaacctacaacataccagaagatcatattttcagcctttcacaccgaactgactgggcatcagacctcaaagccgcgatgggacaggggcagctagatattgtgatcaacaacgcaacgccaagcccagcaattcgcagccttttccaatcaattgcaccaaaaggacgattcgtagaccttacccagcgtctcgatccgtcgttgctcgacccaaggatgttccagcggaacgttactttgtccttggtggattgggagtctttgaccaacccgcagctgggcgccctgatggttagatcactagatttgctacgagcaggggccttaaccccgatcaaggaagaatatatcttttcggtatctgacctgccagaagcattattgtccgtcggccaacagcaacatgagcgagtcgcagcaccggtggtggtggagttctccgcagatgccacagttcctttacttccatctcttcccgcaccacttcaccttaaacccgatgcgacctatattctcgctggggggctgggtgctcttgggcttaccattgcagagaatatgtgtagccacggtgccggccacttggtgttcctcagccgctccggggcatcgagccaaagacagcaagaggcgcttgagagcttccgtgctcgcggatgcaaggtcgacgtggtgaagtgtgacgtgacggaccaagaacaggtgcaagctctggctacccagattcgggagcaatcgtggaatgttcgcggcatcatccaactggcaatggttctccgggactcgatcttcgaaaatatgaccttcgataagtgggaaacagctgtcaaccctaagatcaaaggaacgtggaacctgcacgccgagcttcccaaggatgttgatttcttcatcattctctcttcgctctctggcatcattggaaatactgcccaagctaactactgcgccggtaatacatatgaggacgctcttgcccattataggcgtaagcagggcctcgccgcgacgaccctcaacgtcggcctggtcacggacgcaagccacttcaacgagaattccacaatcgaagactatctgcgtaaatatagtcactggatagccgcccaggtcaccgacagcgagctgcagcacaccatcactgccgtgatgcggaggacagtcggcgacaagaatgagcccgtacccgaccagctcctggtcggtctctcagataacgtccggcgtgacggaaacagtctgaatttgtggccacaggaccgcaaattcgaccacaggatcagtctagaagatgggctcggtgtggttgagaaggatacgaatcaacagaaattaaaggcatccacgacagtcgctcaggcgcacgaggtcgttgagacagcattacgcttgaatgttgccgcggctatgactgcctccccggatgatattgacattgagaaaccgctctatgcctttggcattgactcgcttaaagggattgaagtccgtaattggatttttagcgagctgcaggccgatgtatccgtgtttgaggttcttagtccgatgaccctaagccgcctggctctcaagattgtttcgaagagcacgctcgtgggcgcggagcttgctgcagaagctgcggcggacagtgtggcgtaa

SEQ ID NO: 9 - protein, 2458 aa, Aspergillus nidulans
MNLPGEIHHRFTYMYIAQVSFADLDITKHNLPISTMSSSDSPNYGDATMPIAIVGMAARFSGEATNPSKLWDMMVQGRTGHSAVPENRFDAEAWHHPSHERRGTIQPRSGFFLREDPAVFDAPFFSMTAKEAAGMDPMQRKLLEISYEAFENAGIPITKLPGTATGVYSGVMTNDYELMTAGDPMQLPQNAASGTSRAMLANRISWFYDLRGPSFALDTACSSSLYALHLACQSLQAGETDQALVTGVNLILAPNFISQLSSMHMLSPDGKSHSFDSRANGYARGEALAAVVVKPLYQALADGDTIRAVIRGSGANQDGKTVGITIPNPQAQAELIRKTYATAGLGLEQTGYFEAHGTGTPVGDPIELSAIGTSFGEHRSQNCPLFVGSVKTNVGHTEGAAGLAGVVKTVLALEAGIIPPLADFQELNEKLRLEEWKLALPLKATPWPMPGLRRASVNSFGFGGANAHVILDDAYHYLKSHGLSANHHTTLSESEDSSDSGLEMDSSTSDSGEGQSSKLLLFSAYDGAGIKRTEASWNSHLADILADSKTVDETMGMNDLAYTLSDRRTTFDFRSFAVASSVQDLKAKLENDGLPRLNRASRRSNPVFVFTGQGAQWPAMGRELLSNPIFRASIERSKAVLELEGCEWDVVQVLSDPQDQRIHIPAFSQPVCTILQVALVDLLQSWGIQPAATVGHSSGEVAAAYAAKMISQDEAVRIGYWRGFYSEQVKARLENIRGSMMAVGLSESQATSYLNRVPEGSVVVACINSPSSVTLSGEDHSIKTLEAILQADGHFARKLRVEVAYHSPHMKTVADEFLNAVGIITPQPSEIPMFSSVTETRVEDPATLVASYWMQNLISPVRFSGALATLLNDTPSVKANTRRRRTAGIVWSALIEVGPHEALKGPCRQIMSGLNTKLADQIPYMSVLSRGKSAVETSLTAAGLLWASGHPINIREVNQYRDTGERVITDLPPYPWNHEKGFWHEPAASISARLRKEPRNDLLGVPVAQQNPFERCWQNYLSVSECPWQKDHVITGTVLYPGAGHLIMAFEAAIRLAADNRPLKGVSFSDVHFDKGLVIPSDDHGVETRLCTRPHESLLDWYHYTLYSINATGDWTKHSWGSFSLHYEDAVSVQQAKRSKGEYDDINTRACRKLDVESFYEQLLSIGTEYGPTFRNLVHAAAAPGYHSGVGTITIPDTKSVMPHEFEYPHLIHPATLDAIFHLIFVAMGEGNALSESAIPTRVDRIYISTDLPRGVGAKYTGYGRAEPVSSRDTLGTIVVSDENWSAGPKIIVEGMTVTEVSAGASTSFNSLLIPGGQGRIATLEWKEDVDSLVGPTAESWLAQKGPSIGGQASDVTEAVQRLDAWLELSCFKSTDLGTLVICPSKLKGSFELVKKYGSKHGERYRFGRTTIIEFSENDISAAESAFAPHGIESSYAAIDLSATPEHAMEQLGMFDLIIAEENVIVQFPDVTKILHREGRVAIIRSHALPDERHFAATKGLLKEISFESQDGSILQIAGLGLEMDPAIRSLDDVVLLQHVDASPAAKNFEKRLTAQLTSLGAHVRSNTIANASSLSGNIVISLLEIDCQFVISWTSEEFEQFRQLTNARYVLWITRGGLLDADRASLDYAPSTGLLRTVRVEKPQIRLPHLDLSPSLDLNSDRAVEIVISAFHSSIKPSVKEKNLEMEYAESNGLLYIPRARGHAALDHELALRGEKVSSIPGPLSAPGIARRLETSLAGSPSQARWVPDETVGDKLADFDVEIQVSHVGLEHSKVENYLNGKQLSLAPGLGRVAVGTLTRAGAKVSRFIPGDQVFALHAAPFHTHLRVTEDAVHAVPDILSPAQAAHLPLAAARAWHSLIDVAAFRAGQSVFVNGASDTVGRLTVELARLLKGDVFASVSSDDEKHTLTKTYNIPEDHIFSLSHRTDWASDLKAAMGQGQLDIVINNATPSPAIRSLFQSIAPKGRFVDLTQRLDPSLLDPRMFQRNVTLSLVDWESLTNPQLGALMVRSLDLLRAGALTPIKEEYIFSVSDLPEALLSVGQQQHERVAAPVVVEFSADATVPLLPSLPAPLHLKPDATYILAGGLGALGLTIAENMCSHGAGHLVFLSRSGASSQRQQEALESFRARGCKVDVVKCDVTDQEQVQALATQIREQSWNVRGIIQLAMVLRDSIFENMTFDKWETAVNPKIKGTWNLHAELPKDVDFFIILSSLSGIIGNTAQANYCAGNTYEDALAHYRRKQGLAATTLNVGLVTDASHFNENSTIEDYLRKYSHWIAAQVTDSELQHTITAVMRRTVGDKNEPVPDQLLVGLSDNVRRDGNSLNLWPQDRKFDHRISLEDGLGVVEKDTNQQKLKASTTVAQAHEVVETALRLNVAAAMTASPDDIDIEKPLYAFGIDSLKGIEVRNWIFSELQADVSVFEVLSPMTLSRLALKIVSKSTLVGAELAAEAAADSVA

SEQ ID NO: 10 - protein, 284 aa, Aspergillus nidulans
MPKILCLHGYGTSASILQHQLGPFMAAADPSYEFVFLEGEIECQKAQGLGPFVKGPFLCYNESFAPADIQESCDLIDEMIQAAGPFDGIIGFSQGGSVALSYLLQRQIDGHPPPFRWAVFFSTVIAFAPNDTFGSNILANLTDHEIRLLDGYPATDLSSLHPLTRALCETTAQTFYSAKTGGFISPNTPIAEFSKRDDPSQPRVFHPALLGDRIPIPTVHITGRKDNSLMVGLSVLVQGLCDQRLIRSLTHSGGHNVPRSADDVRAAWAAVDWAIQHSQKQHIW

SEQ ID NO: 11 - DNA, 904 bp, Aspergillus nidulans
  source 1..904: mol_type = genomic DNA
atgcccaagatactttgcttacacggatacgggacatctgcctcgatcctccagcatcagctgggccccttcatggcagctgctgatcccagttatgagttcgtcttcctggaaggagagatagaatgccaaaaggcccaaggtccgtacccttgtcagagcaaggtcacatccaagtaacgagattgcaggactggggcccttcgtgaaggggccattcctatgctacaacgagagcttcgcgccagctgacatccaggagtcctgcgaccttatcgacgagatgatacaagcagccggcccgttcgacgggataatcggctttagccagggcggctcggtcgccctttcgtatctgctccaacgccagatcgacggacacccccctccctttcggtgggccgtgtttttctctacggtgatcgcatttgctcccaatgacacctttggtagcaacatacttgccaatcttacggaccacgagatccgtctgttggatgggtatcctgctacggatctctcgtcgcttcatccgctgacccgagccctatgcgagacaactgcgcagactttctactctgccaaaaccggggggtttatctcccctaataccccaatcgcggagttctctaagcgggatgatccctctcagccgcgagtcttccacccagccttgttgggggaccggattcctattccgactgtgcatattaccggccggaaggacaatagcttgatggtgggtctctctgtccttgtacagggcctatgtgatcagcgtctgatacgatccttgactcattccggtggccacaatgtcccccggtctgcggatgatgtccgtgctgcatgggctgctgtggattgggctatccaacatagtcagaagcagcatatctggtga

SEQ ID NO: 12 - DNA, 855 bp, Aspergillus nidulans
  source 1..855: mol_type = genomic DNA
atgcccaagatactttgcttacacggatacgggacatctgcctcgatcctccagcatcagctgggccccttcatggcagctgctgatcccagttatgagttcgtcttcctggaaggagagatagaatgccaaaaggcccaaggactggggcccttcgtgaaggggccattcctatgctacaacgagagcttcgcgccagctgacatccaggagtcctgcgaccttatcgacgagatgatacaagcagccggcccgttcgacgggataatcggctttagccagggcggctcggtcgccctttcgtatctgctccaacgccagatcgacggacacccccctccctttcggtgggccgtgtttttctctacggtgatcgcatttgctcccaatgacacctttggtagcaacatacttgccaatcttacggaccacgagatccgtctgttggatgggtatcctgctacggatctctcgtcgcttcatccgctgacccgagccctatgcgagacaactgcgcagactttctactctgccaaaaccggggggtttatctcccctaataccccaatcgcggagttctctaagcgggatgatccctctcagccgcgagtcttccacccagccttgttgggggaccggattcctattccgactgtgcatattaccggccggaaggacaatagcttgatggtgggtctctctgtccttgtacagggcctatgtgatcagcgtctgatacgatccttgactcattccggtggccacaatgtcccccggtctgcggatgatgtccgtgctgcatgggctgctgtggattgggctatccaacatagtcagaagcagcatatctggtga